SEQUENCE LISTING (41 sequences)

SEQ ID NO: 1 - DNA, 5835 bp, Artificial Sequence
  SARS-CoV-2 Nsp3 gene cDNA
gcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggt

SEQ ID NO: 2 - DNA, 44 bp, Artificial Sequence
  Nsp3 gene FIP oligonucleotide
cttgttgaccaacagtttgttgacttcaacctgaagaagagcaa

SEQ ID NO: 3 - DNA, 18 bp, Artificial Sequence
  Nsp3 gene F3 oligonucleotide
ggaatttggtgccacttc

SEQ ID NO: 4 - DNA, 41 bp, Artificial Sequence
  Nsp3 gene BIP oligonucleotide
cggcagtgaggacaatcagacactggtgtaagttccatctc

SEQ ID NO: 5 - DNA, 24 bp, Artificial Sequence
  Nsp3 gene B3 oligonucleotide
ctattcacttcaatagtctgaaca

SEQ ID NO: 6 - DNA, 24 bp, Artificial Sequence
  Nsp3 gene LF oligonucleotide
atcatcatctaaccaatcttcttc

SEQ ID NO: 7 - DNA, 24 bp, Artificial Sequence
  Nsp3 gene LB oligonucleotide
tcaaacaattgttgaggttcaacc

SEQ ID NO: 8 - DNA, 21 bp, Artificial Sequence
  Nsp3 gene F1c oligonucleotide
tgttgaccaacagtttgttga

SEQ ID NO: 9 - DNA, 21 bp, Artificial Sequence
  Nsp3 gene F2 oligonucleotide
cttcaacctgaagaagagcaa

SEQ ID NO: 10 - DNA, 22 bp, Artificial Sequence
  Nsp3 gene B1c oligonucleotide
cggcagtgaggacaatcagaca

SEQ ID NO: 11 - DNA, 19 bp, Artificial Sequence
  Nsp3 gene B2 oligonucleotide
ctggtgtaagttccatctc

SEQ ID NO: 12 - DNA, 151 bp, Artificial Sequence
  Nsp3 gene synthetic nucleic acid
cttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccag

SEQ ID NO: 13 - DNA, 3822 bp, Artificial Sequence
  SARS-CoV-2 S gene cDNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 14 - DNA, 50 bp, Artificial Sequence
  S gene FIP oligonucleotide
cgatttgtctgacttcatcacctctaaatgatctctgctttactaatgtc

SEQ ID NO: 15 - DNA, 21 bp, Artificial Sequence
  S gene F3 oligonucleotide
tgttatggagtgtctcctact

SEQ ID NO: 16 - DNA, 38 bp, Artificial Sequence
  S gene BIP oligonucleotide
ctccagggcaaactggaaagcaagctataacgcagcct

SEQ ID NO: 17 - DNA, 22 bp, Artificial Sequence
  S gene B3 oligonucleotide
ccaaccttagaatcaagattgt

SEQ ID NO: 18 - DNA, 25 bp, Artificial Sequence
  S gene F1c oligonucleotide
cgatttgtctgacttcatcacctct

SEQ ID NO: 19 - DNA, 25 bp, Artificial Sequence
  S gene F2 oligonucleotide
aaatgatctctgctttactaatgtc

SEQ ID NO: 20 - DNA, 20 bp, Artificial Sequence
  S gene B1c oligonucleotide
ctccagggcaaactggaaag

SEQ ID NO: 21 - DNA, 18 bp, Artificial Sequence
  S gene B2 oligonucleotide
caagctataacgcagcct

SEQ ID NO: 22 - DNA, 147 bp, Artificial Sequence
  S gene synthetic nucleic acid
aaatgatctctgctttactaatgtctatgcagattcatttgtaattagaggtgatgaagtcagacaaatcgctccagggcaaactggaaagattgctgattataattataaattaccagatgattttacaggctgcgttatagcttg

SEQ ID NO: 23 - DNA, 1260 bp, Artificial Sequence
  SARS-CoV-2 N gene cDNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 24 - DNA, 630 bp, Artificial Sequence
  SARS-CoV-2 N gene 3′ end cDNA
gctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 25 - DNA, 41 bp, Artificial Sequence
  N gene FIP oligonucleotide
tgcggccaatgtttgtaatcagccaaggaaattttggggac

SEQ ID NO: 26 - DNA, 18 bp, Artificial Sequence
  N gene F3 oligonucleotide
aacacaagctttcggcag

SEQ ID NO: 27 - DNA, 39 bp, Artificial Sequence
  N gene BIP oligonucleotide
cgcattggcatggaagtcactttgatggcacctgtgtag

SEQ ID NO: 28 - DNA, 22 bp, Artificial Sequence
  N gene B3 oligonucleotide
gaaatttggatctttgtcatcc

SEQ ID NO: 29 - DNA, 19 bp, Artificial Sequence
  N gene LF oligonucleotide
ttccttgtctgattagttc

SEQ ID NO: 30 - DNA, 18 bp, Artificial Sequence
  N gene LB oligonucleotide
accttcgggaacgtggtt

SEQ ID NO: 31 - DNA, 22 bp, Artificial Sequence
  N gene F1c oligonucleotide
tgcggccaatgtttgtaatcag

SEQ ID NO: 32 - DNA, 19 bp, Artificial Sequence
  N gene F2 oligonucleotide
ccaaggaaattttggggac

SEQ ID NO: 33 - DNA, 20 bp, Artificial Sequence
  N gene B1c oligonucleotide
cgcattggcatggaagtcac

SEQ ID NO: 34 - DNA, 19 bp, Artificial Sequence
  N gene B2 oligonucleotide
tttgatggcacctgtgtag

SEQ ID NO: 35 - DNA, 169 bp, Artificial Sequence
  N gene synthetic nucleic acid
ccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaa

SEQ ID NO: 36 - DNA, 41 bp, Artificial Sequence
  human RNaseP FIP oligonucleotide
gtgtgaccctgaagactcggttttagccactgactcggatc

SEQ ID NO: 37 - DNA, 17 bp, Artificial Sequence
  human RNaseP F3 oligonucleotide
ttgatgagctggagcca

SEQ ID NO: 38 - DNA, 45 bp, Artificial Sequence
  human RNaseP BIP oligonucleotide
cctccgtgatatggctcttcgtttttttcttacatggctctggtc

SEQ ID NO: 39 - DNA, 18 bp, Artificial Sequence
  human RNaseP B3 oligonucleotide
caccctcaatgcagagtc

SEQ ID NO: 40 - DNA, 20 bp, Artificial Sequence
  human RNaseP LF oligonucleotide
atgtggatggctgagttgtt

SEQ ID NO: 41 - DNA, 20 bp, Artificial Sequence
  human RNaseP LB oligonucleotide
catgctgagtactggacctc

(19) United States
(12) Patent Application Publication
(10) Pub. No.: US 2022/0056542 A1
(43) Pub. Date: Feb. 24, 2022

(54) Rapid Viral Diagnostic Test

(71) Applicant: NantCell, Inc., Culver City, CA (US)

(72) Inventors: Hermes GARBÁN, Los Angeles, CA (US); Kayvan NIAZI, Agoura Hills, CA (US); Shahrooz RABIZADEH, Culver City, CA (US); Patrick SOON-SHIONG, Los Angeles, CA (US)

(21) Appl. No.: 17/401,079

(22) Filed: Aug. 12, 2021

Related U.S. Application Data
(60) Provisional application No. 63/069,598, filed on Aug. 24, 2020.
(60) Provisional application No. 63/067,033, filed on Aug. 18, 2020.
(60) Provisional application No. 63/115,127, filed on Nov. 18, 2020.

(51) Int. Cl. C12Q 1/70

(52) U.S. Cl. CPC C12Q 1/701

(57) ABSTRACT

A method for detecting severe acute respiratory syndrome coronavirus 2 (SARS-CoV-2) in a sample is provided. The method includes contacting at least two pairs of primers with a complementary deoxyribonucleic acid (cDNA) derived from SARS-CoV-2 ribonucleic acid (RNA) in the sample; amplifying a portion of the cDNA by quantitative loop-mediated isothermal amplification (qLAMP) in the presence of a marker that exhibits a detectable signal as the cDNA is amplified, wherein the portion of the cDNA comprises a portion of non-structural protein 3 (Nsp3)-gene cDNA, a portion of S-gene cDNA, a portion of a 3′ end of N-gene cDNA, or combinations thereof; periodically measuring the detectable signal during the isothermally amplifying; and determining at least one of an amplification threshold breach or an amplification rate corresponding to levels of the detectable signal versus amplification time. Treatment methods, reaction mixtures, and assay kits are also provided.

### CROSS-REFERENCE TO RELATED APPLICATIONS

This application claims the benefit of U.S. Provisional Application No. 63/067,033, filed on 18 Aug. 2020, and U.S. Provisional Application No. 63/069,598, filed on 24 Aug. 2020, both of which are hereby incorporated by reference in their entirety.

### REFERENCE TO A SEQUENCE LISTING

This application contains references to amino acid sequences and/or nucleic acid sequences which have been submitted concurrently herewith as the sequence listing text file entitled “PAT005229US015 sequences.TXT”, file size 23.1 KiloBytes (KB), created on 25 Sep. 2020. The aforementioned sequence listing is hereby incorporated by reference in its entirety pursuant to 37 C.F.R. § 1.52(e)(5).

### FIELD OF THE INVENTION

The invention relates to assays for detecting severe acute respiratory syndrome coronavirus 2 (SARS-CoV-2) in a sample, and methods of treating the same.

### BACKGROUND

Corona virus disease 2019 (COVID-19) is a pandemic, which according to the Center for Systems Science and Engineering (CSSE) at Johns Hopkins University, has been identified in over 31,940,000 cases and attributed to deaths of over 975,000 people worldwide. First identified in Wuhan, China in December 2019, COVID-19 is caused by severe acute respiratory syndrome coronavirus 2 (SARS-CoV-2).

SARS-CoV-2 is an enveloped, non-segmented, positive sense ribonucleic acid (RNA) virus. Its RNA genome includes RNA sequences that translate into an orf1ab polyprotein, a surface glycoprotein (“Spike”), an orf3a protein, an envelope protein (E protein), a membrane glycoprotein, an orf6 protein, an orf7a protein, an orf8 protein, a nucleocapsid (Nc) phosphoprotein (“nucleoprotein”), and an orf10 protein. The orf1ab polyprotein includes non-structural protein (Nsp) 1, Nsp2, Nsp3, Nsp4, Nsp5, Nsp6, Nsp7, Nsp8, Nsp9, Nsp10, RNA-directed RNA polymerase, helicase, guanine-N7 methyltransferase, uridylate-specific endoribonuclease, and 2′-O-methyltransferase.

Diagnostic testing for human patients for SARS-CoV-2 typically involves performing quantitative reverse transcription polymer chain reaction (qRT-PCR) on a clinical sample. The qRT-PCR requires first processing the sample with a reverse transcriptase to form a complementary DNA (cDNA) molecule, digesting the SARS-CoV-2 RNA with RNaseH, and then performing RT-PCR, which includes lengthy denaturation, annealing, and extension steps. This whole process can take hours to complete and generates backlogs of patients waiting for results. Accordingly, alternative diagnostic testing methods for SARS-CoV-2 are needed to provide fast and accurate results to patients in need.

### SUMMARY

One aspect of the current technology relates to a method for detecting severe acute respiratory syndrome coronavirus 2 (SARS-CoV-2) in a sample. The method comprises contacting at least two pairs of primers with a cDNA derived from SARS-CoV-2 RNA in the sample, and amplifying a portion of the cDNA by quantitative loop-mediated isothermal amplification (qLAMP) in the presence of a marker that exhibits a detectable signal as the cDNA is amplified, wherein the portion of the cDNA comprises a portion of non-structural protein 3 (Nsp3)-gene cDNA, a portion of S-gene cDNA, a portion of a 3′ end of N-gene cDNA, or a combination thereof. The sample includes extracted or non-extracted SARS-CoV-2 RNA. A viral load of about 1000 copies/mL or great in the sample may be quantitatively measured.

In certain aspects, the portion of the cDNA comprises the portion of Nsp3-gene cDNA, which in certain variations, results from the at least two pairs of primers comprising a first primer pair including first and second oligonucleotides having the sequences as set forth in SEQ ID NOs:2 and 3, respectively, and a second primer pair including third and fourth oligonucleotides having the sequences as set forth in SEQ ID NOs:4 and 5, respectively. In some embodiments, the at least two pairs of primers further comprise a third primer pair including fifth and sixth oligonucleotides having the sequences as set forth in SEQ ID NOs:6 and 7, respectively.

In certain aspects, the portion of the cDNA comprises the portion of S-gene cDNA, which in certain variations, results from the at least two pairs of primers comprising a first primer pair including first and second oligonucleotides having the sequences as set forth in SEQ ID NOs:14 and 15, respectively, and a second primer pair including third and fourth oligonucleotides having the sequences as set forth in SEQ ID NOs:16 and 17, respectively.

In certain aspects, the portion of the cDNA comprises the portion of N-gene cDNA, which in certain variations, results from the at least two pairs of primers comprising a first primer pair including first and second oligonucleotides having the sequences as set forth in SEQ ID NOs:25 and 26, respectively, and a second primer pair including third and fourth oligonucleotides having the sequences as set forth in SEQ ID NOs:27 and 28, respectively. In some embodiments, the at least two pairs of primers further comprise a third primer pair including fifth and sixth oligonucleotides having the sequences as set forth in SEQ ID NOs:29 and 30, respectively.

The method may also comprise periodically measuring the detectable signal during the isothermally amplifying, and determining at least one of an amplification threshold breach or an amplification rate corresponding to levels of the detectable signal versus amplification time. In certain embodiments, the isothermally amplifying may be performed for less than or equal to about 60 minutes. In one aspect, the marker may be a light-emitting dye that becomes quenched as the cDNA is amplified. In another aspect, the marker may be a dye that emits detectable light as the cDNA is amplified.

Another aspect of the current technology relates to a method for treating COVID-19 in a subject in need thereof. The method comprises treating the subject with a COVID-19 treatment when a sample taken from the subject demonstrates an amplification detection unit threshold breach and an amplification rate of greater than or equal to about 50,000 detection units per cycle after RNA from or in the sample is combined with a reverse transcriptase, a deoxyribonucleic acid (DNA) polymerase, deoxyribonucleotide triphosphates (dNTPs), a marker, and at least two pairs of primers to form a reaction mixture, and the reaction mixture is subjected to qLAMP. The qLAMP generates amplicons comprising a portion of SARS-CoV-2 Nsp3-gene cDNA, a portion of SARS-CoV-2 S-gene cDNA, a portion of a 3′ end of SARS-CoV-2 N-gene cDNA, or a combination thereof.

In certain aspects, the at least two pairs of primers comprise (1) a first primer pair including first and second oligonucleotides having the sequences as set forth in SEQ ID NOs:2 and 3, respectively, and (2) a second primer pair including third and fourth oligonucleotides having the sequences as set forth in SEQ ID NOs:4 and 5, respectively. In one variation, the at least two pairs of primers further comprise a third primer pair including fifth and sixth oligonucleotides having the sequences as set forth in SEQ ID NOs:6 and 7, respectively.

In certain aspects, the at least two pairs of primers comprise (1) a first primer pair including first and second oligonucleotides having the sequences as set forth in SEQ ID NOs:14 and 15, respectively, and (2) a second primer pair including third and fourth oligonucleotides having the sequences as set forth in SEQ ID NOs:16 and 17, respectively.

In certain aspects, the at least two pairs of primers comprise (1) a first primer pair including first and second oligonucleotides having the sequences as set forth in SEQ ID NOs:25 and 26, respectively, and (2) a second primer pair including third and fourth oligonucleotides having the sequences as set forth in SEQ ID NOs:27 and 28, respectively. In one variation, the at least two pairs of primers further comprise a third primer pair including fifth and sixth oligonucleotides having the sequences as set forth in SEQ ID NOs:29 and 30, respectively.

Another aspect of the current technology relates to a reaction mixture comprising human messenger ribonucleic acid (mRNA), a DNA polymerase configured for loop-mediated isothermal amplification (LAMP), a reverse transcriptase, dNTPs, and at least two pairs of primers configured to amplify a portion of SARS-CoV-2 cDNA selected from the group consisting of a portion of Nsp3-gene cDNA, a portion of S-gene cDNA, a portion of a 3′ end of N-gene cDNA, and a combination thereof. In some variations, the reaction mixture may also comprise at least one of a marker that provides a detectable signal as DNA amplifies, or SARS-CoV-2 RNA.

In certain aspects, the at least two pairs of primers comprise (1) a first primer pair including first and second oligonucleotides having the sequences as set forth in SEQ ID NOs:2 and 3, respectively, and (2) a second primer pair including third and fourth oligonucleotides having the sequences as set forth in SEQ ID NOs:4 and 5, respectively. In a variation, the at least two pairs of primers further comprise a third primer pair including fifth and sixth oligonucleotides having the sequences as set forth in SEQ ID NOs:6 and 7, respectively.

In certain aspects, the at least two pairs of primers comprise (1) a first primer pair including first and second oligonucleotides having the sequences as set forth in SEQ ID NOs:14 and 15, respectively, and (2) a second primer pair including third and fourth oligonucleotides having the sequences as set forth in SEQ ID NOs:16 and 17, respectively.

In certain aspects, the at least two pairs of primers comprise (1) a first primer pair including first and second oligonucleotides having the sequences as set forth in SEQ ID NOs:25 and 26, respectively, and (2) a second primer pair including third and fourth oligonucleotides having the sequences as set forth in SEQ ID NOs:27 and 28, respectively. In a variation, the at least two pairs of primers further comprise a third primer pair including fifth and sixth oligonucleotides having the sequences as set forth in SEQ ID NOs:29 and 30, respectively.

Yet another aspect of the current technology relates to a reaction mixture comprising SARS-CoV-2 cDNA, a DNA polymerase configured for LAMP, dNTPs, and greater than or equal to about 100 amplicons corresponding to a portion of SARS-CoV-2 Nsp3-gene cDNA, a portion of SARS-CoV-2 S-gene cDNA, a portion of a 3′ end of SARS-CoV-2 N-gene cDNA, or a combination thereof. In various embodiments, the amplicons correspond to: (a) the portion of SARS-CoV-2 Nsp3-gene and include a sequence as set forth in SEQ ID NO:12, (b) the portion of SARS-CoV-2 S-gene and include a sequence as set forth in SEQ ID NO:22, (c) the portion of SARS-CoV-2 N-gene and include a sequence as set forth in SEQ ID NO:35, or (d) a combination thereof.

A further aspect of the current technology relates to a diagnostic SARS-CoV-2 assay kit. The assay kit comprises a primer set selected from the group consisting of (a) a first primer pair including oligonucleotides having the sequences as set forth in SEQ ID NOs:2 and 3, and a second primer pair including oligonucleotides having the sequences as set forth in SEQ ID NOs:4 and 5; (b) a primer pair including oligonucleotides having the sequences as set forth in SEQ ID NOs:6 and 7; (c) a first primer pair including oligonucleotides having the sequences as set forth in SEQ ID NOs:14 and 15, and a second primer pair including oligonucleotides having the sequences as set forth in SEQ ID NOs:16 and 17; (d) a first primer pair including oligonucleotides having the sequences as set forth in SEQ ID NOs:25 and 26, and a second primer pair including oligonucleotides having the sequences as set forth in SEQ ID NOs:27 and 28; (e) a primer pair including oligonucleotides having the sequences as set forth in SEQ ID NOs:29 and 30; and (f) a combination thereof.

In certain aspects, the assay kit also comprises control primers configured to amplify human RNaseP. In some variations, the control primers may comprise a first control primer pair including first and second control oligonucleotides having the sequences as set forth in SEQ ID NOs:36 and 37, respectively, and a second control primer pair including third and fourth control oligonucleotides having the sequences as set forth in SEQ ID NOs:38 and 39, respectively. In various embodiments, the control primers may further comprise a third control primer pair including fifth and sixth oligonucleotides having the sequences as set forth in SEQ ID NOs:40 and 41, respectively.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a flow chart describing a qLAMP reaction.

FIG. 2 shows exemplary qLAMP results demonstrating a high SARS-CoV-2 viral load.

FIG. 3 shows exemplary qLAMP results demonstrating a low SARS-CoV-2 viral load.

FIG. 4 shows exemplary qLAMP results demonstrating the presence of human cDNA and an absence of SARS-CoV-2 RNA or cDNA.

FIGS. 5A-5B show positive qLAMP results obtained from reaction mixtures including clinical samples and Nsp3-gene primers (FIG. 5A) and qLAMP control results (FIG. 5B) in accordance with various aspects of the current technology.

FIG. 6 shows qLAMP results obtained from reaction mixtures containing various loads of SARS-CoV-2 genomic DNA and SARS-CoV-2 Nsp3-gene primers in accordance with various aspects of the current technology.

FIG. 7 shows qLAMP results obtained from reaction mixtures containing various dilutions of a SARS-CoV-2 clinical sample and SARS-CoV-2 Nsp3-gene primers in accordance with various aspects of the current technology.

FIG. 8 shows qLAMP results obtained from reaction mixtures containing various loads of a clinical SARS-CoV-2 sample and SARS-CoV-2 Nsp3-gene primers in accordance with various aspects of the current technology. Results obtained from phenol red color changes and SYTO™ 9 fluorescent markers fluorescence are provided.

FIG. 9 shows qLAMP results obtained from reaction mixtures containing SARS-CoV-2 clinical samples and SARS-CoV-2 Nsp3-gene primers in accordance with various aspects of the current technology. Results obtained from phenol red color changes and SYTO™ 9 fluorescent markers fluorescence are provided.

FIG. 10 shows qLAMP results obtained from reaction mixtures containing SARS-CoV-2 clinical samples and SARS-CoV-2 Nsp3-gene primers and S-gene primers in accordance with various aspects of the current technology.

FIG. 11 shows qLAMP results obtained from reaction mixtures containing control SARS-CoV-2 RNA and SARS-CoV-2 Nsp3-gene primers and S-gene primers in accordance with various aspects of the current technology.

FIG. 12 shows qLAMP results obtained from reaction mixtures containing old and new SARS-CoV-2 RNA samples and SARS-CoV-2 Nsp3-gene primers in accordance with various aspects of the current technology.

FIG. 13 shows qLAMP results obtained from reaction mixtures containing 25 copies of SARS-CoV-2 genomic RNA and SARS-CoV-2 Nsp3-gene primers in accordance with various aspects of the current technology.

FIG. 14 shows qLAMP results obtained from reaction mixtures containing extracted and non-extracted SARS-CoV-2 RNA in accordance with various aspects of the current technology.

FIGS. 15A-15D qLAMP results obtained from reaction mixtures containing clinical samples and primers directed to SARS-CoV-2 Nsp-3 gene DNA, SARS-CoV-2 S-gene DNA, and human RNaseP DNA in wells of a 384-well plate having rows A-P in accordance with various aspects of the current technology. FIG. 15A shows the results obtained from the wells of rows A-D, FIG. 15B shows the results obtained from the wells of rows E-H, FIG. 15C shows the results obtained from the wells of rows I-L, and FIG. 15D shows the results obtained from the wells of rows M-P.

### DETAILED DESCRIPTION

The described invention provides methods, compositions, and kits for determining the presence of SARS-CoV-2 in a sample, and methods for treating COVID-19.

### Glossary

The term “expressing” or “expression,” as used herein, means the transcription and translation of a nucleic acid molecule by a cell.

The term “gene,” as used herein, refers to a nucleic acid molecule that encodes a protein or functional RNA (for example, a tRNA). A gene can include regions that do not encode the final protein or RNA product, such as 5′ or 3′ untranslated regions, introns, ribosome binding sites, promoter or enhancer regions, or other associated and/or regulatory sequence regions.

The terms “gene expression” and “expression” are used interchangeably herein to refer to the process by which inheritable information from a gene, such as a DNA sequence, is made into a functional gene product, such as protein or RNA.

The term “hybridizes” refers to the binding of two single stranded nucleic acid molecules to each other through base pairing. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable. Measuring the effects of base incompatibility by quantifying the rate at which two strands anneal can provide information as to the similarity in base sequence between the two strands being annealed

The term “marker” is a probe or reporter that exhibits a detectable signal as an oligonucleotide binds to a template and/or as a nucleic acid molecule is amplified.

The term “nucleic acid,” as used herein, refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide,” as used herein, refers to a chemical compound that consists of a heterocyclic base, a sugar, and one or more phosphate groups. In the most common nucleotides the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. The purines include adenine (A), and guanine (G); the pyrimidines include cytosine (C), thymine (T), and uracil (U).

The term “oligonucleotide” refers a polynucleotide having a small number of nucleotides. The small number of nucleotides is generally less than or equal to 75 nucleotides, but can be greater than this amount to some extent. Oligonucleotides are the basis of primers.

The term “polynucleotide” refers to linear polymeric molecule that is formed from a plurality of nucleotide bases. A polynucleotide is a portion of a nucleic acid molecule.

The term “primer” refers to a nucleic acid molecule which, when hybridized to a strand of DNA or RNA, is capable of serving as the substrate to which nucleotides are added in the synthesis of an extension product in the presence of a suitable polymerization agent (e.g., a DNA polymerase). In some cases, the primer is sufficiently long to uniquely hybridize to a specific region of a DNA or RNA strand. In some cases, the primer is an oligonucleotide and optionally includes a marker.

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “template” refers to a nucleic acid target for nucleic acid synthesis or for a hybridizing olignonucleotide. Templates can be RNA molecules for reverse transcription or RNA or DNA molecules that receive an oligonucleotide for an amplification reaction.

### I. Methods for Determining the Presence of SARS-CoV-2 in a Sample

According to one aspect, the current technology provides a method for determining the presence of SARS-CoV-2 in a sample. More particularly, the method includes determining whether SARS-CoV-2 RNA is present within a sample. SARS-CoV-2 is a positive sense RNA virus have a corresponding genomic cDNA sequence provided by Genbank Accession Number MN908947.3. By reverse transcribing SARS-CoV-2 RNA to generate this corresponding cDNA, detecting a portion of this cDNA, such as a portion of a gene encoded by the cDNA, the presence of SARS-CoV-2 can be indirectly determined.

Isolates of SARS-CoV-2 have been obtained and their analysis has identified genomic differences, especially single nucleotide polymorphisms (SNPs), among the isolates. Some of the isolates may constitute different strains of SARS-CoV-2 having different biological properties. Nonetheless, the current methods are capable of detecting the presence of all SARS-CoV-2 isolates and/or strains that infect and/or cause COVID-19 in human subjects.

The method includes contacting at least two pairs of primers with a cDNA derived from SARS-CoV-2 RNA in a sample and amplifying a portion of the cDNA by quantitative loop-mediated isothermal amplification (qLAMP). In certain aspects, the sample is a biological sample obtained from a human subject. The biological sample can be a biological fluid, a tissue biopsy, or stool. As non-limiting examples, biological fluids include sputum, mucus, saliva, bronchoalveolar lavage fluid, blood, urine, and combinations thereof. The biological samples are obtained with the use of a collection device chosen based on the biological sample to be tested, such as cotton swabs or nylon flocked swabs, e.g., naopharyngeal swabs, oropharyngeal swabs, nasal swabs, and the like, for biological fluids and stool; spatulas, and wood, plastic or metal transfer sticks for stool, and biopsy devices for tissue biopsies, such as fibrobonchoscope brush biopsies of lung tissue, bronchial tissue, and alveolar tissue. In other aspects, the sample can be a sample obtained from cultured bacteria cells, wherein at least a portion of the bacteria cells contain, or are infected with, SARS-CoV-2.

The contacting occurs in a reaction container containing a reaction mixture having a reaction buffer and the at least two pairs of primers. Accordingly, the sample can be directly transferred from the collection device to the reaction mixture. Alternatively, the sample can be transferred from the collection device to a transport medium in a transport container. At or near the time of testing, at least a portion of the transport medium is transferred to the reaction container. In some aspects, the transport container is the reaction container. Therefore, the sample can be transferred from the collection device to a reaction container containing transport medium, wherein the transport medium is the reaction buffer.

Transport media are known in the art, are commercially available, and generally include a salt solution or culture medium, serum, an antibiotic, and an antifugal. The salt solution includes phosphate buffered saline (PBS), Hank's balanced salt solution (HBSS), or the like, at pH 7-8. The culture medium includes Eagle minimal essential medium (E-MEM), Dulbecco's modified Eagle's medium (DMEM), or the like. Antibiotics includes penicillin, ampicillin, vancomycin, kanamycin, streptomycin, gentamicin, the like, and combinations thereof, and an exemplary antifungal is amphotericin. Sera include bovine serum, fetal bovine serum, horse serum, the like, and combinations thereof. Transport media may also include a pH indicator, such as phenol red. An exemplary transport medium includes HBSS pH 7.4, 1% BSA, 15 μg/mL amphotericin, 100 units/mL penicillin, and 50 μg/mL streptomycin. Another exemplary transport medium includes HBSS pH 7.4, 2% fetal bovine serum, 100 μg/mL gentamicin, and 0.5 μg/mL amphotericin.

When obtained from a human subject, the sample includes human DNA and RNA from the subject. SARS-CoV-2 RNA is only present in the sample when the human subject has SARS-CoV-2. Although SARS-CoV-2 RNA can be extracted from the sample using methods and compositions known in the art, such as phenol:chloroform, TRIZOL™ RNA extraction reagent (Thermo Fisher Scientific) and QIAZOL RNA extraction reagent (Qiagen) as non-limiting examples, RNA extraction is not necessary. Accordingly, in certain aspects of the current technology, SARS-CoV-2 RNA is not extracted from the sample.

The reaction mixture includes the reaction buffer, the at least two pairs of primers, a reverse transcriptase, dNTPs, e.g., a solution including dATP, dCTP, dGTP, and dTTP, a marker that provides a detectable signal, and a DNA polymerase. The reaction mixture can further include adjunct agents, such as proteinase K and/or guanidine hydrochloride. However, it is understood that such adjunct agents are not required. Therefore, in some aspects, the reaction mixture is free of, i.e., precludes, adjunct agents such as proteinase K and/or guanidine hydrochloride. The volume of the reaction mixture is greater than or equal to about 15 μL to less than or equal to about 250 μL.

The reaction buffer is typically provided with the DNA polymerase, but generally includes a pH 7-9 buffer, such Tris-HCl, and at least one of (NH₄)₂SO₄, KCl, MgSo₄, or polysorbate 20. The DNA polymerase has high strand displacement activity and lacks exonuclease activity, and that is suitable for isothermal amplification, such as, for example, Bacillus stearothermophilus (Bst) DNA polymerase, Bacillus smithii (Bsm) DNA polymerase, Geobacillus stearothermophilus (Gst) DNA polymerase, Bacillus subtilis phage 29 (phi29) DNA polymerase, SD polymerase, fragments thereof (e.g., large fragment), or derivatives thereof (e.g., mutants with improved performance). The DNA polymerase has an isothermal amplification temperature of greater than or equal to about 30° C. to less than or equal to about 75° C., greater than or equal to about 40° C. to less than or equal to about 70° C., or greater than or equal to about 55° C. to less than or equal to about 65° C. and a deactivation temperature of greater than about 70° C. to less than or equal to about 90° C. Some exemplary commercially available polymerases with high strand displacement include wild-type Bst DNA polymerase, large fragment (New England Biolabs), Bst 2.0 DNA polymerase (New England Biolabs), Bst 2.0 WarmStart™ DNA polymerase (New England Biolabs), Bst 3.0 DNA polymerase (New England Biolabs), phi29 DNA polymerase (New England Biolabs), Bsm DNA polymerase, large fragment (Thermo Fisher Scientific), EquiPhi29™ DNA polymerase (Thermo Fisher Scientific), OmniAmp™ RNA/DNA polymerase (Lucigen), Gst DNA polymerase (Excellgen), and SD DNA polymerase (Bioron). Also, DNA polymerases for LAMP are described by Ignatove et al., BioTechniques (2014) 57:81-87, which is incorporated herein by reference in its entirety. The reverse transcriptase and DNA polymerase are included in the reaction mix at concentrations suggested by their vendors. In some aspects, the buffer is provided in a master mix offered by a vendor of DNA polymerases and reverse transcriptases.

The dNTPs are included in the reaction mixture so that each dNTP (i.e., dATP, dGTP, dCTP, and dTTP) has a final concentration of greater than or equal to about 0.25 mM to less than or equal to about 1.75 mM, or greater than or equal to about 1 mM to less than or equal to about 1.5 mM.

The marker can be any marker that provides a detectable signal in real time. In some aspects the marker is a fluorescent dye that emits detectable light as DNA is amplified. In other aspects, the marker is a light-emitting dye that becomes quenched as DNA is amplified. For example, the marker can be a fluorescent molecule that becomes intercalated in amplifying DNA and emits light while it is intercalated. Alternatively, the marker can be a fluorescing molecule that becomes quenched in amplifying DNA. Non-limiting examples of fluorescent markers include SYTO™ fluorescent markers (Thermo Fisher Scientific), such as SYTO™ 9, 11, 12 13, 14, 15, 16 18, 20, 21, 22, 23, 24, 25, and BC fluorescent markers, SYBR® Green green fluorescent nucleic acid stain (Thermo Fisher Scientific), SYBR® Gold green fluorescent nucleic acid stain (Thermo Fisher Scientific), EvaGreen® green fluorescent nucleic acid stain (Biotium), FAM™ 6-carboxyfluorescein fluorescent dye (Applied Biosystems), TET™ dye phosphoramidite fluorescein dye (Applied Biosystems), VIC® fluorescent dye (Applied Biosystems), HEX™ dye phosphoramidite fluorescent dye (Applied Biosystems), NED™ fluorescent dye (Applied Biosystems), PET® fluorescent dye (Applied Biosystems), JOE™ fluorophores (Lumiprobe), Texas Red® fluorescent dye (Molecular Probes), and combinations thereof. It is understood that additional markers are known in the art and are commercially available. The marker can be free in solution, i.e., not covalently bonded to a molecule comprising DNA, or bonded to a molecule comprising DNA, such as a primer.

The marker can also be a pH indicator or a pH sensitive dye. For example, the pH indicator phenol red is pink-red in color at pHs near neutral, such as from about pH 7.3 to about pH 8. As a neutral solution including phenol red becomes acidic, i.e., as the pH decreases, the phenol red exhibits a sequential color transition from pink-red to orange to yellow. As a neutral solution including phenol red becomes basic, i.e., as the pH increases, the phenol red exhibits a sequential color transition from pink-red to bright pink to fuchsia. Because the reaction mixture has a neutral pH of greater than or equal to about 7.3 to less than or equal to about 8, it exhibits a pink-red color when including phenol red prior to amplification. As DNA accumulates in the reaction mixture, i.e., during amplification, protons are produced, which lower the pH. Consequently, the color of the reaction mixture including the phenol red transitions from pink-red to yellow. This color transition from pink-red to yellow can be visualized by the human eye and serves as an indication that DNA is being amplified.

The marker can be quenched before or after being intercalated into amplifying DNA, or if bonded to a primer, before or after primer binding to a template. Quenching can be achieved by a quenching molecule, for example, by fluorescence resonance energy transfer (FRET). Non-limiting examples of quenching molecules include guanine bases, BHQ®-1 black hole quencher dye (Biosearch Technologies), BHQ®-2 black hole quencher dye (Biosearch Technologies), BHQ®-3 black hole quencher dye (Biosearch Technologies), BLACKBERRY® quencher (BBQ®)-650 quencher dye (Berry and Associates), ECLIPSE® quencher dye (Elitech Group), DABCYL® quencher dye, TAMRA™ fluorescent quencher dye (Applied Biosystems), and combinations thereof.

The cDNA derived from SARS-CoV-2 RNA in the sample is generated by reverse transcribing SARS-CoV-2 genomic RNA using a reverse transcriptase and by methods generally known in the art. The reverse transcriptase, in the presence of dNTPs, synthesizes a cDNA strand complementary to the genomic RNA. As a result an RNA-cDNA hybrid molecule is formed. The RNA strand of the hybrid molecule is removed using, for example, RNase H, resulting in a single stranded cDNA molecule that serves as a template of the qLAMP. Alternatively, the RNA strand can be left intact on the RNA-cDNA hybrid molecule as the DNA polymerase having high strand displacement activity can directly amplify the cDNA strand from the RNA-cDNA hybrid molecule.

The cDNA derived from SARS-CoV-2 RNA includes cDNA sequences corresponding to SARS-CoV-2 ORF1ab (which encodes orf1ab polyprotein), S gene (which encodes surface glycoprotein (“Spike”)), ORF3a (which encodes ORF3a protein), E gene (which encodes envelope protein (E protein)), M gene (which encodes membrane glycoprotein), ORF6 (which encodes ORF6 protein), ORF7a (which encodes ORF7a protein), ORF8 (which encodes ORF8 protein), N gene (which encodes a nucleocapsid (Nc) phosphoprotein (“nucleoprotein”)), ORF10 (which encodes ORF10 protein), and combinations thereof. The orf1ab polyprotein includes non-structural protein (Nsp) 1, Nsp2, Nsp3, Nsp4, Nsp5, Nsp6, Nsp7, Nsp8, Nsp9, Nsp10, RNA-directed RNA polymerase, helicase, guanine-N7 methyltransferase, uridylate-specific endoribonuclease, 2′-O-methyltransferase, and combinations thereof. A portion of the cDNA derived from the SARS-CoV-2 RNA is amplified during the qLAMP.

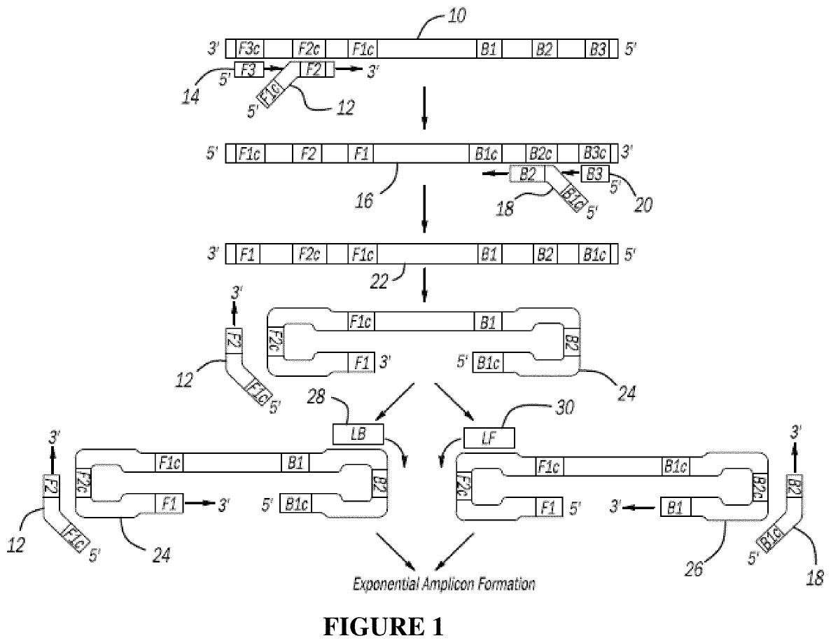

qLAMP requires at least two pairs of primers for isothermal amplification. With reference to FIG. 1, the reaction mixture includes the cDNA derived from SARS-CoV-2 RNA 10 as a template. A first of the at least two pairs of primers includes a primer pair including a forward internal primer oligonucleotide (FIP) 12 and a forward outer primer oligonucleotide (F3) 14. FIP includes a forward 1 complement oligonucleotide sequence (F1c) and a downstream forward 2 oligonucleotide sequence (F2). The F2 portion of FIP binds to a forward 2 complement oligonucleotide sequence (F2c) on the cDNA template 10. The F1c portion of FIP is a complement of a forward 1 oligonucleotide sequence (F1) and does not bind to the cDNA template 10. After elongation of FIP by the DNA polymerase, a first synthetic DNA strand 16 is bonded to the cDNA template 10 except for F1c, which remains unbonded. F3 then binds upstream of F2 at a forward 3 complement oligonucleotide sequence (F3c) on the cDNA template 10 and elongation of F3 by the DNA polymerase causes release of the first synthetic DNA strand 16.

A second of the at least two pairs of primers includes a primer pair including a backward internal primer oligonucleotide (BIP) 18 and a backward outer primer oligonucleotide (B3) 20. BIP includes a backward 1 complement oligonucleotide sequence (B1c) and a downstream backward 2 oligonucleotide sequence (B2). The B2 portion of BIP binds to a backward 2 complement oligonucleotide sequence (B2c) on the first synthetic DNA strand 16. The B1c portion of BIP is a complement of a backward 1 oligonucleotide sequence (B1) and does not bind to the first synthetic DNA strand 16. After elongation of BIP by the DNA polymerase, a second synthetic DNA strand 22 is bonded to the first synthetic DNA strand 16 except for B1c, which remains unbonded. B3 then binds upstream of B2 at a backward 3 complement oligonucleotide sequence (B3c) on the first synthetic DNA strand 16 and elongation of B3 by the DNA polymerase causes release of the second synthetic DNA strand 22.

The second synthetic DNA strand 22 has a 5′ end including B1c, B2, and B1 sequences in a 5′ to 3′ direction. The second synthetic DNA strand 22 also has a 3′ end including F1, F2c, and F1c sequences in a 3′ to 5′ direction. The B1c sequence of the second synthetic DNA strand 22 folds and binds to the B1 sequence and the F1 sequence folds and binds to the F1c sequence. As a result a first dumbbell-shaped synthetic DNA molecule 24 is formed. After another round of elongation of the first and second primer primers 12, 14, 18, 20 on the first dumbbell-shaped synthetic DNA molecule 24, a complementary second dumbbell-shaped synthetic DNA molecule 26 is formed. The at least two pairs of primers including FIP 12, F3 14, BIP 18 and B3 20 bind to the first and second dumbbell-shaped synthetic DNA molecules 24, 26 and amplification proceeds exponentially.

In some aspects, a third of the at least two pairs of primers includes a primer pair including a loop backward oligonucleotide (LB) 28 and a loop forward oligonucleotide (LF) 30. LB and LF 28, 30 bind to loop portions of the first and second dumbbell-shaped synthetic DNA molecules 24, 26, respectively. By including the third pair of primers, exponential amplification is increased.

The F3c, F2c, F1c, B1, B2, and B3 sequences are all located within a DNA sequence that expresses a single SARS-CoV-2 RNA gene. Therefore, the qLAMP generates amplicons including a portion of a SARS-CoV-2 gene cDNA.

In certain aspects of the current technology, the at least two primers bind to a portion of SARS-CoV-2 Nsp3 gene cDNA. The SARS-CoV-2 Nsp3 gene cDNA has the sequence set forth in SEQ ID NO:1. Here, the at least two pairs of primers include a first primer pair including first and second oligonucleotides having the sequences as set forth in SEQ ID NOs: 2 and 3, respectively, and a second primer pair including third and fourth oligonucleotides having the sequences as set forth in SEQ ID NOs:4 and 5, respectively. The at least two pairs of primers can also include a third primer pair including fifth and sixth oligonucleotides having the sequences as set forth in SEQ ID NOs:6 and 7, respectively. The DNA sequences set forth in SEQ ID NOs:2, 3, 4, 5, 6, and 7 correspond to Nsp3 FIP, F3, BIP, B3, LF, and LP oligonucleotide sequences, respectively. SEQ ID NO:2 (Nsp3-FIP) includes oligonucleotides sequences for F1c and F2 as set forth in SEQ ID NOs: 8 and 9, respectively, and SEQ ID NO:4 (Nsp3-BIP) includes oligonucleotides sequences for B1c and B2 as set forth in SEQ ID NOs:10 and 11, respectively. When the at least two pairs of primers include oligonucleotides as set forth in SEQ ID NOs:2-5, and optionally also oligonucleotides as set forth in SEQ ID NOs:6-7, at least a portion of the amplicons resulting from qLAMP include the DNA sequence as set forth in SEQ ID NO:12. The sequences set forth in SEQ ID NOs:1-12 are shown in Table 1.

TABLE 1
DNA sequences according to the current technology.

| SEQ ID NO: | Name | Sequence |
|---|---|---|
| 1 | Nsp3 gene cDNA | GCACCAACAAAGGTTACTTTTGGTGATGACACTGTGATAGA |
| | | AGTGCAAGGTTACAAGAGTGTGAATATCACTTTTGAACTTG |
| | | ATGAAAGGATTGATAAAGTACTTAATGAGAAGTGCTCTGCC |
| | | TATACAGTTGAACTCGGTACAGAAGTAAATGAGTTCGCCTG |
| | | TGTTGTGGCAGATGCTGTCATAAAAACTTTGCAACCAGTAT |
| | | CTGAATTACTTACACCACTGGGCATTGATTTAGATGAGTGG |
| | | AGTATGGCTACATACTACTTATTTGATGAGTCTGGTGAGTT |
| | | TAAATTGGCTTCACATATGTATTGTTCTTTCTACCCTCCAG |
| | | ATGAGGATGAAGAAGAAGGTGATTGTGAAGAAGAAGAGTTT |
| | | GAGCCATCAACTCAATATGAGTATGGTACTGAAGATGATTA |
| | | CCAAGGTAAACCTTTGGAATTTGGTGCCACTTCTGCTGCTC |
| | | TTCAACCTGAAGAAGAGCAAGAAGAAGATTGGTTAGATGAT |
| | | GATAGTCAACAAACTGTTGGTCAACAAGACGGCAGTGAGGA |
| | | CAATCAGACAACTACTATTCAAACAATTGTTGAGGTTCAAC |
| | | CTCAATTAGAGATGGAACTTACACCAGTTGTTCAGACTATT |
| | | GAAGTGAATAGTTTTAGTGGTTATTTAAAACTTACTGACAA |
| | | TGTATACATTAAAAATGCAGACATTGTGGAAGAAGCTAAAA |
| | | AGGTAAAACCAACAGTGGTTGTTAATGCAGCCAATGTTTAC |
| | | CTTAAACATGGAGGAGGTGTTGCAGGAGCCTTAAATAAGGC |
| | | TACTAACAATGCCATGCAAGTTGAATCTGATGATTACATAG |
| | | CTACTAATGGACCACTTAAAGTGGGTGGTAGTTGTGTTTTA |
| | | AGCGGACACAATCTTGCTAAACACTGTCTTCATGTTGTCGG |
| | | CCCAAATGTTAACAAAGGTGAAGACATTCAACTTCTTAAGA |
| | | GTGCTTATGAAAATTTTAATCAGCACGAAGTTCTACTTGCA |
| | | CCATTATTATCAGCTGGTATTTTTGGTGCTGACCCTATACA |
| | | TTCTTTAAGAGTTTGTGTAGATACTGTTCGCACAAATGTCT |
| | | ACTTAGCTGTCTTTGATAAAAATCTCTATGACAAACTTGTT |
| | | TCAAGCTTTTTGGAAATGAAGAGTGAAAAGCAAGTTGAACA |
| | | AAAGATCGCTGAGATTCCTAAAGAGGAAGTTAAGCCATTTA |
| | | TAACTGAAAGTAAACCTTCAGTTGAACAGAGAAAACAAGAT |
| | | GATAAGAAAATCAAAGCTTGTGTTGAAGAAGTTACAACAAC |
| | | TCTGGAAGAAACTAAGTTCCTCACAGAAAACTTGTTACTTT |
| | | ATATTGACATTAATGGCAATCTTCATCCAGATTCTGCCACT |
| | | CTTGTTAGTGACATTGACATCACTTTCTTAAAGAAAGATGC |
| | | TCCATATATAGTGGGTGATGTTGTTCAAGAGGGTGTTTTAA |
| | | CTGCTGTGGTTATACCTACTAAAAAGGCTGGTGGCACTACT |
| | | GAAATGCTAGCGAAAGCTTTGAGAAAAGTGCCAACAGACAA |
| | | TTATATAACCACTTACCCGGGTCAGGGTTTAAATGGTTACA |
| | | CTGTAGAGGAGGCAAAGACAGTGCTTAAAAAGTGTAAAAGT |
| | | GCCTTTTACATTCTACCATCTATTATCTCTAATGAGAAGCA |
| | | AGAAATTCTTGGAACTGTTTCTTGGAATTTGCGAGAAATGC |
| | | TTGCACATGCAGAAGAAACACGCAAATTAATGCCTGTCTGT |
| | | GTGGAAACTAAAGCCATAGTTTCAACTATACAGCGTAAATA |
| | | TAAGGGTATTAAAATACAAGAGGGTGTGGTTGATTATGGTG |
| | | CTAGATTTTACTTTTACACCAGTAAAACAACTGTAGCGTCA |
| | | CTTATCAACACACTTAACGATCTAAATGAAACTCTTGTTAC |
| | | AATGCCACTTGGCTATGTAACACATGGCTTAAATTTGGAAG |
| | | AAGCTGCTCGGTATATGAGATCTCTCAAAGTGCCAGCTACA |
| | | GTTTCTGTTTCTTCACCTGATGCTGTTACAGCGTATAATGG |
| | | TTATCTTACTTCTTCTTCTAAAACACCTGAAGAACATTTTA |
| | | TTGAAACCATCTCACTTGCTGGTTCCTATAAAGATTGGTCC |
| | | TATTCTGGACAATCTACACAACTAGGTATAGAATTTCTTAA |
| | | GAGAGGTGATAAAAGTGTATATTACACTAGTAATCCTACCA |
| | | CATTCCACCTAGATGGTGAAGTTATCACCTTTGACAATCTT |
| | | AAGACACTTCTTTCTTTGAGAGAAGTGAGGACTATTAAGGT |
| | | GTTTACAACAGTAGACAACATTAACCTCCACACGCAAGTTG |
| | | TGGACATGTCAATGACATATGGACAACAGTTTGGTCCAACT |
| | | TATTTGGATGGAGCTGATGTTACTAAAATAAAACCTCATAA |
| | | TTCACATGAAGGTAAAACATTTTATGTTTTACCTAATGATG |
| | | ACACTCTACGTGTTGAGGCTTTTGAGTACTACCACACAACT |
| | | GATCCTAGTTTTCTGGGTAGGTACATGTCAGCATTAAATCA |
| | | CACTAAAAAGTGGAAATACCCACAAGTTAATGGTTTAACTT |
| | | CTATTAAATGGGCAGATAACAACTGTTATCTTGCCACTGCA |
| | | TTGTTAACACTCCAACAAATAGAGTTGAAGTTTAATCCACC |
| | | TGCTCTACAAGATGCTTATTACAGAGCAAGGGCTGGTGAAG |
| | | CTGCTAACTTTTGTGCACTTATCTTAGCCTACTGTAATAAG |
| | | ACAGTAGGTGAGTTAGGTGATGTTAGAGAAACAATGAGTTA |
| | | CTTGTTTCAACATGCCAATTTAGATTCTTGCAAAAGAGTCT |
| | | TGAACGTGGTGTGTAAAACTTGTGGACAACAGCAGACAACC |
| | | CTTAAGGGTGTAGAAGCTGTTATGTACATGGGCACACTTTC |
| | | TTATGAACAATTTAAGAAAGGTGTTCAGATACCTTGTACGT |
| | | GTGGTAAACAAGCTACAAAATATCTAGTACAACAGGAGTCA |
| | | CCTTTTGTTATGATGTCAGCACCACCTGCTCAGTATGAACT |
| | | TAAGCATGGTACATTTACTTGTGCTAGTGAGTACACTGGTA |
| | | ATTACCAGTGTGGTCACTATAAACATATAACTTCTAAAGAA |
| | | ACTTTGTATTGCATAGACGGTGCTTTACTTACAAAGTCCTC |
| | | AGAATACAAAGGTCCTATTACGGATGTTTTCTACAAAGAAA |
| | | ACAGTTACACAACAACCATAAAACCAGTTACTTATAAATTG |
| | | GATGGTGTTGTTTGTACAGAAATTGACCCTAAGTTGGACAA |
| | | TTATTATAAGAAAGACAATTCTTATTTCACAGAGCAACCAA |
| | | TTGATCTTGTACCAAACCAACCATATCCAAACGCAAGCTTC |
| | | GATAATTTTAAGTTTGTATGTGATAATATCAAATTTGCTGA |
| | | TGATTTAAACCAGTTAACTGGTTATAAGAAACCTGCTTCAA |
| | | GAGAGCTTAAAGTTACATTTTTCCCTGACTTAAATGGTGAT |
| | | GTGGTGGCTATTGATTATAAACACTACACACCCTCTTTTAA |
| | | GAAAGGAGCTAAATTGTTACATAAACCTATTGTTTGGCATG |
| | | TTAACAATGCAACTAATAAAGCCACGTATAAACCAAATACC |
| | | TGGTGTATACGTTGTCTTTGGAGCACAAAACCAGTTGAAAC |
| | | ATCAAATTCGTTTGATGTACTGAAGTCAGAGGACGCGCAGG |
| | | GAATGGATAATCTTGCCTGCGAAGATCTAAAACCAGTCTCT |
| | | GAAGAAGTAGTGGAAAATCCTACCATACAGAAAGACGTTCT |
| | | TGAGTGTAATGTGAAAACTACCGAAGTTGTAGGAGACATTA |
| | | TACTTAAACCAGCAAATAATAGTTTAAAAATTACAGAAGAG |
| | | GTTGGCCACACAGATCTAATGGCTGCTTATGTAGACAATTC |
| | | TAGTCTTACTATTAAGAAACCTAATGAATTATCTAGAGTAT |
| | | TAGGTTTGAAAACCCTTGCTACTCATGGTTTAGCTGCTGTT |
| | | AATAGTGTCCCTTGGGATACTATAGCTAATTATGCTAAGCC |
| | | TTTTCTTAACAAAGTTGTTAGTACAACTACTAACATAGTTA |
| | | CACGGTGTTTAAACCGTGTTTGTACTAATTATATGCCTTAT |
| | | TTCTTTACTTTATTGCTACAATTGTGTACTTTTACTAGAAG |
| | | TACAAATTCTAGAATTAAAGCATCTATGCCGACTACTATAG |
| | | CAAAGAATACTGTTAAGAGTGTCGGTAAATTTTGTCTAGAG |
| | | GCTTCATTTAATTATTTGAAGTCACCTAATTTTTCTAAACT |
| | | GATAAATATTATAATTTGGTTTTTACTATTAAGTGTTTGCC |
| | | TAGGTTCTTTAATCTACTCAACCGCTGCTTTAGGTGTTTTA |
| | | ATGTCTAATTTAGGCATGCCTTCTTACTGTACTGGTTACAG |
| | | AGAAGGCTATTTGAACTCTACTAATGTCACTATTGCAACCT |
| | | ACTGTACTGGTTCTATACCTTGTAGTGTTTGTCTTAGTGGT |
| | | TTAGATTCTTTAGACACCTATCCTTCTTTAGAAACTATACA |
| | | AATTACCATTTCATCTTTTAAATGGGATTTAACTGCTTTTG |
| | | GCTTAGTTGCAGAGTGGTTTTTGGCATATATTCTTTTCACT |
| | | AGGTTTTTCTATGTACTTGGATTGGCTGCAATCATGCAATT |
| | | GTTTTTCAGCTATTTTGCAGTACATTTTATTAGTAATTCTT |
| | | GGCTTATGTGGTTAATAATTAATCTTGTACAAATGGCCCCG |
| | | ATTTCAGCTATGGTTAGAATGTACATCTTCTTTGCATCATT |
| | | TTATTATGTATGGAAAAGTTATGTGCATGTTGTAGACGGTT |
| | | GTAATTCATCAACTTGTATGATGTGTTACAAACGTAATAGA |
| | | GCAACAAGAGTCGAATGTACAACTATTGTTAATGGTGTTAG |
| | | AAGGTCCTTTTATGTCTATGCTAATGGAGGTAAAGGCTTTT |
| | | GCAAACTACACAATTGGAATTGTGTTAATTGTGATACATTC |
| | | TGTGCTGGTAGTACATTTATTAGTGATGAAGTTGCGAGAGA |
| | | CTTGTCACTACAGTTTAAAAGACCAATAAATCCTACTGACC |
| | | AGTCTTCTTACATCGTTGATAGTGTTACAGTGAAGAATGGT |
| | | TCCATCCATCTTTACTTTGATAAAGCTGGTCAAAAGACTTA |
| | | TGAAAGACATTCTCTCTCTCATTTTGTTAACTTAGACAACC |
| | | TGAGAGCTAATAACACTAAAGGTTCATTGCCTATTAATGTT |
| | | ATAGTTTTTGATGGTAAATCAAAATGTGAAGAATCATCTGC |
| | | AAAATCAGCGTCTGTTTACTACAGTCAGCTTATGTGTCAAC |
| | | CTATACTGTTACTAGATCAGGCATTAGTGTCTGATGTTGGT |
| | | GATAGTGCGGAAGTTGCAGTTAAAATGTTTGATGCTTACGT |
| | | TAATACGTTTTCATCAACTTTTAACGTACCAATGGAAAAAC |
| | | TCAAAACACTAGTTGCAACTGCAGAAGCTGAACTTGCAAAG |
| | | AATGTGTCCTTAGACAATGTCTTATCTACTTTTATTTCAGC |
| | | AGCTCGGCAAGGGTTTGTTGATTCAGATGTAGAAACTAAAG |
| | | ATGTTGTTGAATGTCTTAAATTGTCACATCAATCTGACATA |
| | | GAAGTTACTGGCGATAGTTGTAATAACTATATGCTCACCTA |
| | | TAACAAAGTTGAAAACATGACACCCCGTGACCTTGGTGCTT |
| | | GTATTGACTGTAGTGCGCGTCATATTAATGCGCAGGTAGCA |
| | | AAAAGTCACAACATTGCTTTGATATGGAACGTTAAAGATTT |
| | | CATGTCATTGTCTGAACAACTACGAAAACAAATACGTAGTG |
| | | CTGCTAAAAAGAATAACTTACCTTTTAAGTTGACATGTGCA |
| | | ACTACTAGACAAGTTGTTAATGTTGTAACAACAAAGATAGC |
| | | ACTTAAGGGTGGT |
| 2 | Nsp3-FIP | CTTGTTGACCAACAGTTTGTTGACTTCAACCTGAAGAAGAG |
| | | CAA |
| 3 | Nsp3-F3 | GGAATTTGGTGCCACTTC |
| 4 | Nsp3-BIP | CGGCAGTGAGGACAATCAGACACTGGTGTAAGTTCCATCTC |
| 5 | Nsp3-B3 | CTATTCACTTCAATAGTCTGAACA |
| 6 | Nsp3-LF | ATCATCATCTAACCAATCTTCTTC |
| 7 | Nsp3-LB | TCAAACAATTGTTGAGGTTCAACC |
| 8 | NSP3-F1c | TGTTGACCAACAGTTTGTTGA |
| 9 | NSP3-F2 | CTTCAACCTGAAGAAGAGCAA |
| 10 | NSP3-B1c | CGGCAGTGAGGACAATCAGACA |
| 11 | NSP3-B2 | CTGGTGTAAGTTCCATCTC |
| 12 | portion of Nsp3 | CTTCAACCTGAAGAAGAGCAAGAAGAAGATTGGTTAGATGA |
| | gene in amplicon | TGATAGTCAACAAACTGTTGGTCAACAAGACGGCAGTGAGG |
| | | ACAATCAGACAACTACTATTCAAACAATTGTTGAGGTTCAA |
| | | CCTCAATTAGAGATGGAACTTACACCAG |
| 13 | S gene cDNA | ATGTTTGTTTTTCTTGTTTTATTGCCACTAGTCTCTAGTCA |
| | | GTGTGTTAATCTTACAACCAGAACTCAATTACCCCCTGCAT |
| | | ACACTAATTCTTTCACACGTGGTGTTTATTACCCTGACAAA |
| | | GTTTTCAGATCCTCAGTTTTACATTCAACTCAGGACTTGTT |
| | | CTTACCTTTCTTTTCCAATGTTACTTGGTTCCATGCTATAC |
| | | ATGTCTCTGGGACCAATGGTACTAAGAGGTTTGATAACCCT |
| | | GTCCTACCATTTAATGATGGTGTTTATTTTGCTTCCACTGA |
| | | GAAGTCTAACATAATAAGAGGCTGGATTTTTGGTACTACTT |
| | | TAGATTCGAAGACCCAGTCCCTACTTATTGTTAATAACGCT |
| | | ACTAATGTTGTTATTAAAGTCTGTGAATTTCAATTTTGTAA |
| | | TGATCCATTTTTGGGTGTTTATTACCACAAAAACAACAAAA |
| | | GTTGGATGGAAAGTGAGTTCAGAGTTTATTCTAGTGCGAAT |
| | | AATTGCACTTTTGAATATGTCTCTCAGCCTTTTCTTATGGA |
| | | CCTTGAAGGAAAACAGGGTAATTTCAAAAATCTTAGGGAAT |
| | | TTGTGTTTAAGAATATTGATGGTTATTTTAAAATATATTCT |
| | | AAGCACACGCCTATTAATTTAGTGCGTGATCTCCCTCAGGG |
| | | TTTTTCGGCTTTAGAACCATTGGTAGATTTGCCAATAGGTA |
| | | TTAACATCACTAGGTTTCAAACTTTACTTGCTTTACATAGA |
| | | AGTTATTTGACTCCTGGTGATTCTTCTTCAGGTTGGACAGC |
| | | TGGTGCTGCAGCTTATTATGTGGGTTATCTTCAACCTAGGA |
| | | CTTTTCTATTAAAATATAATGAAAATGGAACCATTACAGAT |
| | | GCTGTAGACTGTGCACTTGACCCTCTCTCAGAAACAAAGTG |
| | | TACGTTGAAATCCTTCACTGTAGAAAAAGGAATCTATCAAA |
| | | CTTCTAACTTTAGAGTCCAACCAACAGAATCTATTGTTAGA |
| | | TTTCCTAATATTACAAACTTGTGCCCTTTTGGTGAAGTTTT |
| | | TAACGCCACCAGATTTGCATCTGTTTATGCTTGGAACAGGA |
| | | AGAGAATCAGCAACTGTGTTGCTGATTATTCTGTCCTATAT |
| | | AATTCCGCATCATTTTCCACTTTTAAGTGTTATGGAGTGTC |
| | | TCCTACTAAATTAAATGATCTCTGCTTTACTAATGTCTATG |
| | | CAGATTCATTTGTAATTAGAGGTGATGAAGTCAGACAAATC |
| | | GCTCCAGGGCAAACTGGAAAGATTGCTGATTATAATTATAA |
| | | ATTACCAGATGATTTTACAGGCTGCGTTATAGCTTGGAATT |
| | | CTAACAATCTTGATTCTAAGGTTGGTGGTAATTATAATTAC |
| | | CTGTATAGATTGTTTAGGAAGTCTAATCTCAAACCTTTTGA |
| | | GAGAGATATTTCAACTGAAATCTATCAGGCCGGTAGCACAC |
| | | CTTGTAATGGTGTTGAAGGTTTTAATTGTTACTTTCCTTTA |
| | | CAATCATATGGTTTCCAACCCACTAATGGTGTTGGTTACCA |
| | | ACCATACAGAGTAGTAGTACTTTCTTTTGAACTTCTACATG |
| | | CACCAGCAACTGTTTGTGGACCTAAAAAGTCTACTAATTTG |
| | | GTTAAAAACAAATGTGTCAATTTCAACTTCAATGGTTTAAC |
| | | AGGCACAGGTGTTCTTACTGAGTCTAACAAAAAGTTTCTGC |
| | | CTTTCCAACAATTTGGCAGAGACATTGCTGACACTACTGAT |
| | | GCTGTCCGTGATCCACAGACACTTGAGATTCTTGACATTAC |
| | | ACCATGTTCTTTTGGTGGTGTCAGTGTTATAACACCAGGAA |
| | | CAAATACTTCTAACCAGGTTGCTGTTCTTTATCAGGATGTT |
| | | AACTGCACAGAAGTCCCTGTTGCTATTCATGCAGATCAACT |
| | | TACTCCTACTTGGCGTGTTTATTCTACAGGTTCTAATGTTT |
| | | TTCAAACACGTGCAGGCTGTTTAATAGGGGCTGAACATGTC |
| | | AACAACTCATATGAGTGTGACATACCCATTGGTGCAGGTAT |
| | | ATGCGCTAGTTATCAGACTCAGACTAATTCTCCTCGGCGGG |
| | | CACGTAGTGTAGCTAGTCAATCCATCATTGCCTACACTATG |
| | | TCACTTGGTGCAGAAAATTCAGTTGCTTACTCTAATAACTC |
| | | TATTGCCATACCCACAAATTTTACTATTAGTGTTACCACAG |
| | | AAATTCTACCAGTGTCTATGACCAAGACATCAGTAGATTGT |
| | | ACAATGTACATTTGTGGTGATTCAACTGAATGCAGCAATCT |
| | | TTTGTTGCAATATGGCAGTTTTTGTACACAATTAAACCGTG |
| | | CTTTAACTGGAATAGCTGTTGAACAAGACAAAAACACCCAA |
| | | GAAGTTTTTGCACAAGTCAAACAAATTTACAAAACACCACC |
| | | AATTAAAGATTTTGGTGGTTTTAATTTTTCACAAATATTAC |
| | | CAGATCCATCAAAACCAAGCAAGAGGTCATTTATTGAAGAT |
| | | CTACTTTTCAACAAAGTGACACTTGCAGATGCTGGCTTCAT |
| | | CAAACAATATGGTGATTGCCTTGGTGATATTGCTGCTAGAG |
| | | ACCTCATTTGTGCACAAAAGTTTAACGGCCTTACTGTTTTG |
| | | CCACCTTTGCTCACAGATGAAATGATTGCTCAATACACTTC |
| | | TGCACTGTTAGCGGGTACAATCACTTCTGGTTGGACCTTTG |
| | | GTGCAGGTGCTGCATTACAAATACCATTTGCTATGCAAATG |
| | | GCTTATAGGTTTAATGGTATTGGAGTTACACAGAATGTTCT |
| | | CTATGAGAACCAAAAATTGATTGCCAACCAATTTAATAGTG |
| | | CTATTGGCAAAATTCAAGACTCACTTTCTTCCACAGCAAGT |
| | | GCACTTGGAAAACTTCAAGATGTGGTCAACCAAAATGCACA |
| | | AGCTTTAAACACGCTTGTTAAACAACTTAGCTCCAATTTTG |
| | | GTGCAATTTCAAGTGTTTTAAATGATATCCTTTCACGTCTT |
| | | GACAAAGTTGAGGCTGAAGTGCAAATTGATAGGTTGATCAC |
| | | AGGCAGACTTCAAAGTTTGCAGACATATGTGACTCAACAAT |
| | | TAATTAGAGCTGCAGAAATCAGAGCTTCTGCTAATCTTGCT |
| | | GCTACTAAAATGTCAGAGTGTGTACTTGGACAATCAAAAAG |
| | | AGTTGATTTTTGTGGAAAGGGCTATCATCTTATGTCCTTCC |
| | | CTCAGTCAGCACCTCATGGTGTAGTCTTCTTGCATGTGACT |
| | | TATGTCCCTGCACAAGAAAAGAACTTCACAACTGCTCCTGC |
| | | CATTTGTCATGATGGAAAAGCACACTTTCCTCGTGAAGGTG |
| | | TCTTTGTTTCAAATGGCACACACTGGTTTGTAACACAAAGG |
| | | AATTTTTATGAACCACAAATCATTACTACAGACAACACATT |
| | | TGTGTCTGGTAACTGTGATGTTGTAATAGGAATTGTCAACA |
| | | ACACAGTTTATGATCCTTTGCAACCTGAATTAGACTCATTC |
| | | AAGGAGGAGTTAGATAAATATTTTAAGAATCATACATCACC |
| | | AGATGTTGATTTAGGTGACATCTCTGGCATTAATGCTTCAG |
| | | TTGTAAACATTCAAAAAGAAATTGACCGCCTCAATGAGGTT |
| | | GCCAAGAATTTAAATGAATCTCTCATCGATCTCCAAGAACT |
| | | TGGAAAGTATGAGCAGTATATAAAATGGCCATGGTACATTT |
| | | GGCTAGGTTTTATAGCTGGCTTGATTGCCATAGTAATGGTG |
| | | ACAATTATGCTTTGCTGTATGACCAGTTGCTGTAGTTGTCT |
| | | CAAGGGCTGTTGTTCTTGTGGATCCTGCTGCAAATTTGATG |
| | | AAGACGACTCTGAGCCAGTGCTCAAAGGAGTCAAATTACAT |
| | | TACACATAA |
| 14 | S-FIP | CGATTTGTCTGACTTCATCACCTCTAAATGATCTCTGCTTT |
| | | ACTAATGTC |
| 15 | S-F3 | TGTTATGGAGTGTCTCCTACT |
| 16 | S-BIP | CTCCAGGGCAAACTGGAAAGCAAGCTATAACGCAGCCT |
| 17 | S-B3 | CCAACCTTAGAATCAAGATTGT |
| 18 | S-F1c | CGATTTGTCTGACTTCATCACCTCT |
| 19 | S-F2 | AAATGATCTCTGCTTTACTAATGTC |
| 20 | S-B1c | CTCCAGGGCAAACTGGAAAG |
| 21 | S-B2 | CAAGCTATAACGCAGCCT |
| 22 | portion of S gene | AAATGATCTCTGCTTTACTAATGTCTATGCAGATTCATTTG |
| | cDNA in amplicon | TAATTAGAGGTGATGAAGTCAGACAAATCGCTCCAGGGCAA |
| | | ACTGGAAAGATTGCTGATTATAATTATAAATTACCAGATGA |
| | | TTTTACAGGCTGCGTTATAGCTTG |
| 23 | N gene cDNA | ATGTCTGATAATGGACCCCAAAATCAGCGAAATGCACCCCG |
| | | CATTACGTTTGGTGGACCCTCAGATTCAACTGGCAGTAACC |
| | | AGAATGGAGAACGCAGTGGGGCGCGATCAAAACAACGTCGG |
| | | CCCCAAGGTTTACCCAATAATACTGCGTCTTGGTTCACCGC |
| | | TCTCACTCAACATGGCAAGGAAGACCTTAAATTCCCTCGAG |
| | | GACAAGGCGTTCCAATTAACACCAATAGCAGTCCAGATGAC |
| | | CAAATTGGCTACTACCGAAGAGCTACCAGACGAATTCGTGG |
| | | TGGTGACGGTAAAATGAAAGATCTCAGTCCAAGATGGTATT |
| | | TCTACTACCTAGGAACTGGGCCAGAAGCTGGACTTCCCTAT |
| | | GGTGCTAACAAAGACGGCATCATATGGGTTGCAACTGAGGG |
| | | AGCCTTGAATACACCAAAAGATCACATTGGCACCCGCAATC |
| | | CTGCTAACAATGCTGCAATCGTGCTACAACTTCCTCAAGGA |
| | | ACAACATTGCCAAAAGGCTTCTACGCAGAAGGGAGCAGAGG |
| | | CGGCAGTCAAGCCTCTTCTCGTTCCTCATCACGTAGTCGCA |
| | | ACAGTTCAAGAAATTCAACTCCAGGCAGCAGTAGGGGAACT |
| | | TCTCCTGCTAGAATGGCTGGCAATGGCGGTGATGCTGCTCT |
| | | TGCTTTGCTGCTGCTTGACAGATTGAACCAGCTTGAGAGCA |
| | | AAATGTCTGGTAAAGGCCAACAACAACAAGGCCAAACTGTC |
| | | ACTAAGAAATCTGCTGCTGAGGCTTCTAAGAAGCCTCGGCA |
| | | AAAACGTACTGCCACTAAAGCATACAATGTAACACAAGCTT |
| | | TCGGCAGACGTGGTCCAGAACAAACCCAAGGAAATTTTGGG |
| | | GACCAGGAACTAATCAGACAAGGAACTGATTACAAACATTG |
| | | GCCGCAAATTGCACAATTTGCCCCCAGCGCTTCAGCGTTCT |
| | | TCGGAATGTCGCGCATTGGCATGGAAGTCACACCTTCGGGA |
| | | ACGTGGTTGACCTACACAGGTGCCATCAAATTGGATGACAA |
| | | AGATCCAAATTTCAAAGATCAAGTCATTTTGCTGAATAAGC |
| | | ATATTGACGCATACAAAACATTCCCACCAACAGAGCCTAAA |
| | | AAGGACAAAAAGAAGAAGGCTGATGAAACTCAAGCCTTACC |
| | | GCAGAGACAGAAGAAACAGCAAACTGTGACTCTTCTTCCTG |
| | | CTGCAGATTTGGATGATTTCTCCAAACAATTGCAACAATCC |
| | | ATGAGCAGTGCTGACTCAACTCAGGCCTAA |
| 24 | 3′ end of N gene | GCTGGCAATGGCGGTGATGCTGCTCTTGCTTTGCTGCTGCT |
| | cDNA | TGACAGATTGAACCAGCTTGAGAGCAAAATGTCTGGTAAAG |
| | | GCCAACAACAACAAGGCCAAACTGTCACTAAGAAATCTGCT |
| | | GCTGAGGCTTCTAAGAAGCCTCGGCAAAAACGTACTGCCAC |
| | | TAAAGCATACAATGTAACACAAGCTTTCGGCAGACGTGGTC |
| | | CAGAACAAACCCAAGGAAATTTTGGGGACCAGGAACTAATC |
| | | AGACAAGGAACTGATTACAAACATTGGCCGCAAATTGCACA |
| | | ATTTGCCCCCAGCGCTTCAGCGTTCTTCGGAATGTCGCGCA |
| | | TTGGCATGGAAGTCACACCTTCGGGAACGTGGTTGACCTAC |
| | | ACAGGTGCCATCAAATTGGATGACAAAGATCCAAATTTCAA |
| | | AGATCAAGTCATTTTGCTGAATAAGCATATTGACGCATACA |
| | | AAACATTCCCACCAACAGAGCCTAAAAAGGACAAAAAGAAG |
| | | AAGGCTGATGAAACTCAAGCCTTACCGCAGAGACAGAAGAA |
| | | ACAGCAAACTGTGACTCTTCTTCCTGCTGCAGATTTGGATG |
| | | ATTTCTCCAAACAATTGCAACAATCCATGAGCAGTGCTGAC |
| | | TCAACTCAGGCCTAA |
| 25 | N-FIP | TGCGGCCAATGTTTGTAATCAGCCAAGGAAATTTTGGGGAC |
| 26 | N-F3 | AACACAAGCTTTCGGCAG |
| 27 | N-BIP | CGCATTGGCATGGAAGTCACTTTGATGGCACCTGTGTAG |
| 28 | N-B3 | GAAATTTGGATCTTTGTCATCC |
| 29 | N-LF | TTCCTTGTCTGATTAGTTC |
| 30 | N-LB | ACCTTCGGGAACGTGGTT |
| 31 | N-F1c | TGCGGCCAATGTTTGTAATCAG |
| 32 | N-F2 | CCAAGGAAATTTTGGGGAC |
| 33 | N-B1c | CGCATTGGCATGGAAGTCAC |
| 34 | N-B2 | TTTGATGGCACCTGTGTAG |
| 35 | portion of N gene | CCAAGGAAATTTTGGGGACCAGGAACTAATCAGACAAGGAA |
| | cDNA in amplicon | CTGATTACAAACATTGGCCGCAAATTGCACAATTTGCCCCC |
| | | AGCGCTTCAGCGTTCTTCGGAATGTCGCGCATTGGCATGGA |
| | | AGTCACACCTTCGGGAACGTGGTTGACCTACACAGGTGCCA |
| | | TCAAA |
| 36 | RNaseP-FIP | GTGTGACCCTGAAGACTCGGTTTTAGCCACTGACTCGGATC |
| 37 | RNaseP-F3 | TTGATGAGCTGGAGCCA |
| 38 | RNaseP-BIP | CCTCCGTGATATGGCTCTTCGTTTTTTTCTTACATGGCTCT |
| | | GGTC |
| 39 | RNaseP-B3 | CACCCTCAATGCAGAGTC |
| 40 | RNaseP-LF | ATGTGGATGGCTGAGTTGTT |
| 41 | RNaseP-LB | CATGCTGAGTACTGGACCTC |

In certain other aspects of the current technology, the at least two primers bind to a portion of SARS-CoV-2 S gene cDNA. The SARS-CoV-2 S gene cDNA has the sequence set forth in SEQ ID NO:13. Here, the at least two pairs of primers include a first primer pair including first and second oligonucleotides having the sequences as set forth in SEQ ID NOs:14 and 15, respectively, and a second primer pair including third and fourth oligonucleotides having the sequences as set forth in SEQ ID NOs:16 and 17, respectively. The DNA sequences set forth in SEQ ID NOs:14, 15, 16, and 17 correspond to S gene FIP, F3, BIP, and B3 oligonucleotide sequences, respectively. SEQ ID NO:14 (S-FIP) includes oligonucleotides sequences for S-F1c and S-F2 as set forth in SEQ ID NOs: 18 and 19, respectively, and SEQ ID NO:16 (Nsp3-BIP) includes oligonucleotides sequences for B1c and B2 as set forth in SEQ ID NOs:20 and 21, respectively. When the at least two pairs of primers include oligonucleotides as set forth in SEQ ID NOs:14-17, at least a portion of the amplicons resulting from qLAMP include the DNA sequence as set forth in SEQ ID NO:22. The sequences set forth in SEQ ID NOs:13-22 are shown in Table 1.

In yet other certain aspects of the current technology, the at least two primers bind to a portion of a 3′ end of SARS-CoV-2 N gene cDNA. The SARS-CoV-2 N gene cDNA has the sequence set forth in SEQ ID NO:23. The 3′ end of the N gene includes a 3′ half of the N gene cDNA. Inasmuch as SEQ ID NO:23 includes 1260 bases, the 3′ end of the N gene cDNA includes the final 630 bases of the N gene cDNA on the 3′ end as set forth in SEQ ID NO:24. Here, the at least two pairs of primers include a first primer pair including first and second oligonucleotides having the sequences as set forth in SEQ ID NOs:25 and 26, respectively, and a second primer pair including third and fourth oligonucleotides having the sequences as set forth in SEQ ID NOs:27 and 28, respectively. The at least two pairs of primers can also include a third primer pair including fifth and sixth oligonucleotides having the sequences as set forth in SEQ ID NOs:29 and 30, respectively. The DNA sequences set forth in SEQ ID NOs:25, 26, 27, 28, 29, and 30 correspond to N gene FIP, F3, BIP, B3, LF, and LP oligonucleotide sequences, respectively. SEQ ID NO:25 (N-FIP) includes oligonucleotides sequences for N-F1c and N-F2 as set forth in SEQ ID NOs: 31 and 32, respectively, and SEQ ID NO:27 (N-BIP) includes oligonucleotides sequences for B1c and B2 as set forth in SEQ ID NOs:33 and 34, respectively. When the at least two pairs of primers include oligonucleotides as set forth in SEQ ID NOs:25-28, and optionally also oligonucleotides as set forth in SEQ ID NOs:29-30, at least a portion of the amplicons resulting from qLAMP include the DNA sequence as set forth in SEQ ID NO:35. The sequences set forth in SEQ ID NOs:23-35 are shown in Table 1.

The above-described oligonucleotides have a high selectivity for SARS-CoV-2. For example, a BLAST analysis performed on the SARS-CoV-2 oligonucleotides versus reference sequences from the entire genomes of other similar and/or common viruses, namely Human Coronavirus 229E, Human Coronavirus OC43, Human Coronavirus HKU1, Human Coronavirus NL63, SARS-CoV, MERS-CoV, Adenovirus, stain ad71, Human Metapneumovirus, Parainfluenza virus 1, stain washington/1967, parainfluenza virus 2, stain GREER, parainfluenza virus 3, stain HPIV3/MEX/1526/2005, parainfluenza virus 4, stain M-25, Influenza A (H1N1), Influenza A (H3N2), Influenza B (Victoria), Influenza B (yamagata), Enterovirus D68(EV-D68), Respiratory syncytial virus, and Human rhinovirus 14, provides the percent identities shown in Table 2 for Nsp3 gene oligonucleotides, Table 3 for S gene oligonucleotides, and Table 4 for N gene oligonucleotides. As shown in Tables 2 and 3, none of the Nsp3-gene or S-gene oligonucleotides has greater than an 80% match with any of the comparative virus genomes. As can be seen in Table 4, only 3 N-gene oligonucleotides have greater than a 90% match with only the SARS-CoV genome. Therefore, the probability of cross-reactivity between the Nsp3-gene, S-gene, and N-gene oligonucleotides with other viruses is very low.

TABLE 2
Percent identity of SARS-CoV-2 Nsp3-gene oligonucleotides versus the entire
genomes of comparative viruses.

| | | F1c | F2 | F3 | B1c | B2 | B3 | LF | LB |
|---|---|---|---|---|---|---|---|---|---|
| | | (SEQ ID | (SEQ ID | (SEQ ID | (SEQ ID | (SEQ ID | (SEQ ID | (SEQ ID | (SEQ ID |
| Virus | GenBank | NO: 8) | NO: 2) | NO: 3) | NO: 4) | NO: 4) | NO: 5) | NO: 6) | NO: 7) |
| SARS-CoV-2 | MN908947.3 | 100% | 100% | 100% | 100% | 100% | 100% | 100% | 100% |
| Human Coronavirus 229E | NC_002645.1 | 52.50% | 47.60% | 65.40% | 60% | 57.60% | 73.30% | 54.50% | 54% |
| Human Coronavirus | NC_006213.1 | 58.30% | 51.30% | 69.20% | 50% | 77.30% | 53.80% | 70% | 51.10% |
| OC43 | | | | | | | | | |
| Human Coronavirus | NC_006577.2 | 54.10% | 65.50% | 54.50% | 70.40% | 39% | 59% | 63.90% | 62.10% |
| HKU1 | | | | | | | | | |
| Human Coronavirus | NC_005831.2 | 74.10% | 59.30% | 70.80% | 60.60% | 70.80% | 53.50% | 53.50% | 59.50% |
| NL63 | | | | | | | | | |
| SARS CoV | NC_004718.3 | 64.50% | 55.30% | 72% | 56.80% | 61.30% | 60.50% | 78.60% | 62.20% |
| MERS CoV | NC_019843.3 | 70% | 50% | 56.70% | 43.50% | 73.90% | 55.80% | 55.80% | 65.70% |
| Adenovirus, stain ad71 | X67709.1 | 52.60% | 57.60% | 44.70% | 56.80% | 53.10% | 58.30% | 58.30% | 65.50% |
| Human Metapneumovirus | NC_039199.1 | 50% | 63.60% | 45.70% | 51.30% | 55.90% | 70% | 55% | 55% |
| Parainfluenza virus 1, | AF457102.1 | 52.60% | 71.40% | 50% | 58.30% | 59.40% | 64.70% | 65.70% | 63.90% |
| stain washington/1967 | | | | | | | | | |
| parainfluenza virus 2, | AF533012.1 | 61.80% | 76% | 56.70% | 62.50% | 55.90% | 53.80% | 54.80% | 57.90% |
| stain GREER | | | | | | | | | |
| parainfluenza virus 3, stain | KF530234.1 | 67.90% | 52.80% | 68% | 54.30% | 53.10% | 57.90% | 64.75% | 56.40% |
| HPIV3/MEX/1526/2005 | | | | | | | | | |
| parainfluenza virus 4, | NC_021928.1 | 53.40% | 52.50% | 58.60% | 58.80% | 64.30% | 63.90% | 46.30% | 61.10% |
| stain M-25 | | | | | | | | | |
| Influenza A (H1N1) | FJ966079.1 | 79.20% | 57.10% | 54.50% | 48.70% | 55.90% | 55% | 48.80% | 53.80% |
| Influenza A (H3N2) | KT002533.1 | 50% | 59.40% | 57.10% | 48.80% | 56.20% | 48.80% | 60% | 48.90% |
| Influenza B (Victoria) | MN230203.1 | 48.60% | 63.30% | 55.20% | 55.30% | 48.70% | 55% | 58.30% | 59.40% |
| Influenza B (yamagata) | MK715533.1 | 52.90% | 60.60% | 60% | 50% | 63% | 64.70% | 62.90% | 48% |
| Enterovirus D68(EV- | KP745766.1 | 51.40% | 72.40% | 58.10% | 65.50% | 69.20% | 47.60% | 55.30% | 50% |
| D68) | | | | | | | | | |
| Respiratory syncytial virus | U39661.1 | 58.80% | 70.40% | 53.10% | 58.80% | 56.70% | 55% | 58.30% | 57.50% |
| Human rhinovirus 14 | NC_001490.1 | 47.40% | 60.60% | 56.70% | 63.60% | 62.10% | 55% | 53.50% | 51.20% |

TABLE 3
Percent identity of SARS-CoV-2 S-gene oligonucleotides versus
the entire genomes of comparative viruses.

| | | F1c | F2 | F3 | B1c | B2 | B3 |
|---|---|---|---|---|---|---|---|
| | | (SEQ ID | (SEQ ID | (SEQ ID | (SEQ ID | (SEQ ID | (SEQ ID |
| Virus | GenBank | NO: 18) | NO: 19) | NO: 15) | NO: 20) | NO: 21) | NO: 17) |
| SARS-CoV-2 | MN908947.3 | 100% | 100% | 100% | 100% | 100% | 100% |
| Human Coronavirus 229E | NC_002645.1 | 62% | 62.90% | 68% | 57.10% | 62.10% | 69% |
| Human Coronavirus OC43 | NC_006213.1 | 59% | 57.10% | 61% | 67.90% | 60% | 72.40% |
| Human Coronavirus HKU1 | NC_006577.2 | 60% | 72.70% | 73.10% | 58.10% | 58.10% | 64.50% |
| Human Coronavitus NL63 | NC_005831.2 | 51.10% | 69.70% | 56.80% | 55.90% | 81.10% | 67.70% |
| SARS CoV | NC_004718.3 | 57.10% | 62.20% | 60% | 59.40% | 60% | 52.50% |
| MERS CoV | NC_019843.3 | 58.50% | 53.30% | 74.10% | 61.30% | 77.30% | 71.40% |
| Adenovirus, stain ad71 | X67709.1 | 41.10% | 45.70% | 51.40% | 44.70% | 61.50% | 57.60% |
| Human Metapneumovitus | NC_039199.1 | 58.30% | 47.10% | 55.90% | 69.20% | 59.30% | 53.80% |
| Parainfluenza virus 1, stain | AF457102.1 | 51.10% | 59.50% | 56.80% | 62.10% | 55.20% | 58.20% |
| washington/1967 | | | | | | | |
| parainfluenza virus 2, stain | AF533012.1 | 52.40% | 60.50% | 55.30% | 58.60% | 64% | 52.80% |
| GREER | | | | | | | |
| parainfluenza virus 3, stain | KF530234.1 | 52.40% | 51.10% | 50% | 57.60% | 53.10% | 59.40% |
| HPIV3/MEX/1526/2005 | | | | | | | |
| parainfluenza virus 4, stain M-25 | NC_021928.1 | 52.20% | 53% | 63.30% | 63.30% | 51.10% | 60.60% |
| Influenza A (H1N1) | FJ966079.1 | 54.10% | 59.30% | 45% | 58.10% | 41% | 64.50% |
| Influenza A (H3N2) | KT002533.1 | 64.50% | 61.10% | 55.90% | 61.30% | 50% | 51.20% |
| Influenza B (Victoria) | MN230203.1 | 55% | 51.20% | 55.90% | 48.60% | 54.80% | 51.40% |
| Influenza B (yamagata) | MK715533.1 | 57.50% | 51.20% | 60.60% | 53.10% | 48.50% | 51.40% |
| Enterovirus D68(EV-D68) | KP745766.1 | 53.30% | 55.60% | 62.50% | 53.30% | 57.70% | 62.50% |
| Respiratory syncytial virus | U39661.1 | 51.20% | 58.60% | 60.60% | 58.80% | 59.30% | 59.40% |
| Human rhinovirus 14 | NC_001490.1 | 53.50% | 56.40% | 59.40% | 55.60% | 65.40% | 67.70% |

TABLE 4
Percent identity of SARS-CoV-2 N-gene oligonucleotides versus
the entire genomes of comparative viruses.

| | | F1c | F2 | F3 | B1c | B2 | B3 | LF | LB |
|---|---|---|---|---|---|---|---|---|---|
| | | (SEQ ID | (SEQ ID | (SEQ ID | (SEQ ID | (SEQ ID | (SEQ ID | (SEQ ID | (SEQ ID |
| Virus | GenBank | NO: 31) | NO: 32) | NO: 26) | NO: 33) | NO: 34) | NO: 28) | NO: 29) | NO: 30) |
| SARS-CoV-2 | MN908947.3 | 100% | 100% | 100% | 100% | 100% | 100% | 100% | 100% |
| Human Coronavirus 229E | NC_002645.1 | 61.80% | 48.60% | 68% | 51.40% | 66.70% | 63.60% | 62.10% | 60% |
| Human Coronavirus | NC_006213.1 | 51.30% | 67.90% | 63% | 61.30% | 62.10% | 55.60% | 52.80% | 55.20% |
| OC43 | | | | | | | | | |
| Human Coronavirus | NC_006577.2 | 51.30% | 72% | 56.70% | 48.70% | 48.40% | 51.40% | 58.60% | 63% |
| HKU1 | | | | | | | | | |
| Human Coronavirus | NC_005831.2 | 70% | 56.20% | 60.70% | 51.40% | 58.10% | 51.40% | 66.70% | 63% |
| NL63 | | | | | | | | | |
| SARS CoV | NC_004718.3 | 57.90% | 90% | 62.10% | 100% | 56.20% | 91.30% | 76% | 73.90% |
| MERS CoV | NC_019843.3 | 53.80% | 51.50% | 64.30% | 62.50% | 55.90% | 53.70% | 57.60% | 65.40% |
| Adenovirus, stain ad71 | X67709.1 | 60% | 35.60% | 51.50% | 51.40% | 60% | 59.40% | 47.40% | 47.10% |
| Human Metapneumovitus | NC_039199.1 | 56.80% | 65.40% | 56.20% | 52.60% | 64.30% | 53.70% | 72% | 60.70% |
| Parainfluenza virus 1, | AF457102.1 | 58.30% | 48.60% | 63.30% | 63.30% | 50% | 57.90% | 65.50% | 63% |
| stain washington/1967 | | | | | | | | | |
| parainfluenza virus 2, | AF533012.1 | 57.90% | 54.80% | 54.80% | 59.40% | 69.02% | 70% | 56.20% | 60.70% |
| stain GREER | | | | | | | | | |
| parainfluenza virus 3, stain | KF530234.1 | 55.30% | 62.10% | 60.70% | 56.70% | 72% | 53.80% | 59.40% | 52.90% |
| HPIV3/MEX/1526/2005 | | | | | | | | | |
| parainfluenza virus 4, | NC_021928.1 | 58.30% | 55.90% | 47.10% | 54.50% | 66.70% | 56.80% | 57.60% | 44.70% |
| stain M-25 | | | | | | | | | |
| Influenza A (H1N1) | FJ966079.1 | 48.80% | 56.20% | 47.20% | 52.08% | 59.40% | 66.70% | 63.30% | 61.50% |
| Influenza A (H3N2) | KT002533.1 | 50% | 57.60% | 52.90% | 48.70% | 53.10% | 35.40% | 63.30% | 58.60% |
| Influenza B (Victoria) | MN230203.1 | 63.03% | 58.10% | 48.60% | 66.70% | 70.08% | 51.20% | 55.90% | 56.70% |
| Influenza B (yamagata) | MK715533.1 | 63.30% | 70.80% | 48.60% | 66.70% | 46.20% | 48.70% | 64.30% | 40.05% |
| Enterovirus D68(EV-D68) | KP745766.1 | 52.50% | 51.40% | 63% | 55.60% | 69.20% | 58.80% | 64.30% | 47.20% |
| Respiratory syncytial | U39661.1 | 54.30% | 59.40% | 52.90% | 54.50% | 59.40% | 67.70% | 55.90% | 58.60% |
| virus | | | | | | | | | |
| Human rhinovirus 14 | NC_001490.1 | 58.80% | 52.90% | 70.80% | 58.10% | 52.90% | 57.90% | 65.50% | 70.80% |

The at least two pairs of primers are included in the reaction mixture at independent concentrations of greater than or equal to about 0.1 μM to less than or equal to about 1 mM, or greater than or equal to about 1 μM to less than or equal to about 1.75 μM. In some aspects of the current technology, the FIB and BIP primers each have a concentration of greater than or equal to about 1 μM to less than or equal to about 2 μM, the F3 and B3 primers each have a concentration of greater than or equal to about 0.1 μM to less than or equal to about 0.3 μM, and the optional LF and LB primers each have a concentration of greater than or equal to about 0.3 μM to less than or equal to about 0.5 μM.

The at least two pairs of primers individually include oligonucleotides as discussed above, and can further include a marker. For example, the marker can be bonded to the 5′ end or the 3′ end of the oligonucleotides. Upon binding of the at least two pairs of primers to a template, the marker either emits light or becomes quenched. In other aspects, the at least two pairs of primers individually include oligonucleotides as discussed above, and can further include the marker and a quencher. For example, the marker can be bonded to one of the 5′ end or the 3′ end and the quencher can be bonded to the other of the 5′ end or the 3′ end. Upon binding of the at least two pairs of primers to a template, the marker may emit light, such as by being released from the primer and being spatially separated from the quencher. Because the at least two pairs of primers can individually include oligonucleotides and a marker, it is possible to perform multiplex reactions for detecting two or more SARS-CoV-2 genes in the sample. For example, at least two primers directed to a first SARS-CoV-2 gene cDNA can include a first marker having a first detectable signal and at least two different primers directed to a second SARS-CoV-2 gene cDNA can include a second marker having a second detectable signal, wherein the first detectable signal is different form the second detectable signal. As a non-limiting example, the first detectable signal can be emitted light having a first wavelength, i.e., a first color, and the second detectable signal can be emitted light having a second wavelength, i.e., a second color. By monitoring the detectable signals, the presence of both SARS-CoV-2 genes can be determined.

In order to ensure the sample is processed properly, a control reaction mixture can also be prepared. As discussed above, when the sample is obtained from a human subject, the sample includes the human subject's nucleic acids, which include at least one of DNA or mRNA. Therefore, amplifying and determining the presence of the subject's nucleic acids by qLAMP serves as a positive control. Accordingly, the control reaction mixture may include at least two pairs of control primers configured to amplify a DNA corresponding to a human gene of the subject, such as cDNA that expresses ribonuclease P (RNaseP). Here, the control primers comprise a first control primer pair including first and second oligonucleotides having the sequences as set forth in SEQ ID NOs:36 and 37, respectively, and a second control primer pair including third and fourth oligonucleotides having the sequences as set forth in SEQ ID NOs:38 and 39, respectively. The at least two pairs of control primers can also include a third control primer pair including fifth and sixth oligonucleotides having the sequences as set forth in SEQ ID NOs:40 and 41, respectively. The DNA sequences set forth in SEQ ID NOs:36, 37, 38, 39, 40, and 41 correspond to human RNaseP FIP, F3, BIP, B3, LF, and LP cDNA oligonucleotide sequences, respectively. Therefore, the at least two control primers include oligonucleotides as set forth in SEQ ID NOs:36-39, and optionally also oligonucleotides as set forth in SEQ ID NOs:40-41 and result in amplicons having a sequence corresponding to human RNaseP cDNA. It is understood that the at least two pairs of control primers can be directed to a human DNA sequence other than cDNA corresponding to RNaseP. The sequences set forth in SEQ ID NOs:36-41 are shown in Table 1.

The qLAMP is performed by heating the reaction mixture to an isothermal amplification temperature and maintaining the isothermal amplification temperature for a period of time. The isothermal amplification temperature is chosen in view of the DNA polymerase used, but is generally greater than or equal to about 30° C. to less than or equal to about 75° C., greater than or equal to about 40° C. to less than or equal to about 70° C., or greater than or equal to about 55° C. to less than or equal to about 65° C. The period of time can be predetermined, i.e., selected prior to beginning the qLAMP, or determined during the qLAMP. For example, the qLAMP may be terminated after a clear and unambiguous positive or negative result is obtained. Nonetheless, the period of time is generally greater than or equal to about 10 minutes to less than or equal to about 2 hours, greater than or equal to about 10 minutes to less than or equal to about 1 hour, or greater than or equal to about 10 minutes to less than or equal to about 30 minutes.

Prior to the isothermal amplification, the method optionally includes denaturing double stranded DNA or RNA-DNA hybrid molecules by heating the reaction mixture to a denaturing temperature and maintaining the denaturing temperature for greater than or equal to about 1 minute to less than or equal to about 10 minutes. The deactivation temperature is greater than about 85° C. to less than or equal to about 100° C.

After the isothermal amplification, the method optionally includes deactivating the DNA polymerase by heating the reaction mixture to a deactivation temperature and maintaining the deactivation temperature for greater than or equal to about 1 minute to less than or equal to about 10 minutes. The deactivation temperature is greater than about 70° C. to less than or equal to about 90° C., with the proviso that the deactivation temperature is at least about 5° C. higher than the isothermal amplification temperature.

After the isothermal amplification or the optional deactivation, the method can also optionally include storing the reaction mixture by cooling the reaction mixture to storage temperature and maintaining the storage temperature for greater than or equal to about 1 minute to less than or equal to about 24 hours or longer. The storage temperature is greater than about 0° C. to less than or equal to about 20° C.

The method may further include periodically measuring the detectable signal provided by the marker and determining at least one of an amplification threshold breach or an amplification rate corresponding to levels of the detectable signal versus amplification time. Periodically measuring includes detecting and measuring the detectable signal after constant and equal time intervals while the qLAMP is being performed. The constant and equal time intervals can be seconds or minutes. For example, the periodically measuring the detectable signal can be performed every 10 seconds, every 15 seconds, every 20 seconds, every 25 seconds, every 30 seconds, every 35 seconds, every 40 seconds, every 45 seconds, every 50 seconds, every 55 seconds, every 1 minute, every 1.5 minutes, every 2 minutes, every 2.5 minutes, every 3 minutes, every 3.5 minutes, every 4 minutes, every 4.5 minutes, every 5 minutes and so on for a total reaction time of greater than or equal to about 10 minutes to less than or equal to about 2 hours, or greater than or equal to about 10 minutes to less than or equal to about 1 hour. As a non-limiting example, the detectable signal is fluorescence emitted by a marker, wherein the fluorescence increases as DNA amplifies. The periodically measuring can be performed by measuring relative fluorescence units (RFUs) every minute for about 1 hour, wherein every minute constitutes a cycle. Thus, 60 cycles are performed over the about 1 hour and measuring and detecting is performed every cycle (or minute).

The amplification threshold is determined relative to a baseline, the baseline being an average detectable signal, e.g., RFUs, calculated form the first five cycles of the qLAMP. More particularly, the amplification threshold is 100%, i.e., 2×, the baseline. The amplification rate is the slope of a graph of detectable signal (e.g., in RFUs) versus time. As such, when the detectable signal is light emitted by a marker, the amplification rate can have the units RFU/time interval (e.g., RFU/minute). A sample is determined to be positive for SARS-CoV-2 when the amplification threshold is breached and the amplification rate at the amplification threshold plus three cycles (in the above example, plus three minutes) is greater than or equal to 50,000 detectable units/minute, or 50,000 RFU/minute for the fluorescent marker. A sample is determined to be negative for SARS-CoV-2 when the amplification rate at the amplification threshold plus three cycles is less than 50,000 or when the threshold is not breached. A summary of an exemplary threshold and amplification rate interpretation is provided in Table 5. Therefore, the method may include determining at least one of an amplification threshold breach or an amplification rate corresponding to levels of the detectable signal versus amplification time

TABLE 5
Threshold and amplification rate interpretation for marker that
emits light during elongation and a cycle time of 1 minute.

| Threshold Breach (>100% | Amplification Rate at | |
|---|---|---|
| of baseline)? | Breach + 3 Cycles | Interpretation |
| Yes | ≥50,000 | Positive |
| Yes | <50,000 | Negative |
| No | Any | Negative |

The method is suitable for detecting, and optionally quantitatively measuring, a SARS-CoV-2 viral load in the reaction mixture of greater than or equal to about 1000 copies/mL, greater than or equal to about 500 copies/mL, greater than or equal to about 250 copies/mL, greater than or equal to about 100 copies/mL, or greater than or equal to about 50 copies/mL. Moreover, in some aspects of the current technology, two or more targets can be amplified in a multiplex reaction in a single reaction tube. For example, a reaction mixture can include at least two pairs of SARS-CoV-2 primers directed to one SARS-CoV-2 cDNA and at least two pairs of control primers directed to a human cDNA, such as human RNaseP cDNA, wherein the at least two pairs of SARS-CoV-2 primers include a first marker and the at least two pairs of control primers include a second marker, wherein the first and second markers exhibit different detectable signals. The first and second markers can be periodically measured as discussed above. In another example, a reaction mixture can include a first primer set including at least two pairs of SARS-CoV-2 primers directed to a first SARS-CoV-2 cDNA, a second primer set including at least two pairs of SARS-CoV-2 primers directed to a second SARS-CoV-2 cDNA, and optionally a control primer set including at least two pairs of control primers directed to a human cDNA, such as human RNaseP cDNA, wherein the first primer set, second primer set, and optional control primer set have markers that exhibit different detectable signals. Each of the markers can be periodically measured as discussed above.

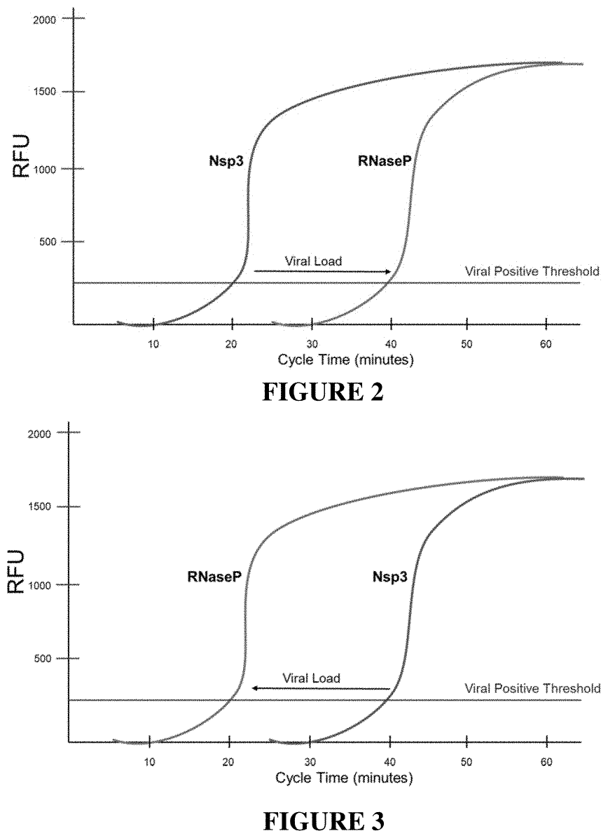

Moreover, by comparing a qLAMP curve resulting from SARS-CoV-2 primers with a qLAMP curve resulting from human control primers, a viral load can be determined to be high or low. FIG. 2 shows exemplary qLAMP results showing amplification resulting from primers directed to SARS-CoV-2 Nsp3-gene cDNA and primers directed to human RNaseP cDNA. In this example, the SARS-CoV-2 Nsp3 primers resulted in a threshold breach about 20 minutes/cycles before the threshold breach resulting from the human RNaseP primers. The earlier detection of SARS-CoV-2 cDNA can be attributed to a higher SARS-CoV-2 cDNA content relative to the human mRNA content. Accordingly, a high viral load is determined.

FIG. 3 also shows exemplary qLAMP results showing amplification resulting from primers directed to SARS-CoV-2 Nsp3-gene cDNA and primers directed to human RNaseP cDNA. In this example, the human RNaseP primers resulted in a threshold breach about 20 minutes/cycles before the threshold breach resulting from the human SARS-CoV-2 Nsp3-gene primers. The later detection of SARS-CoV-2 cDNA can be attributed to a lower SARS-CoV-2 cDNA content relative to the human mRNA content. Accordingly, a low viral load is determined.

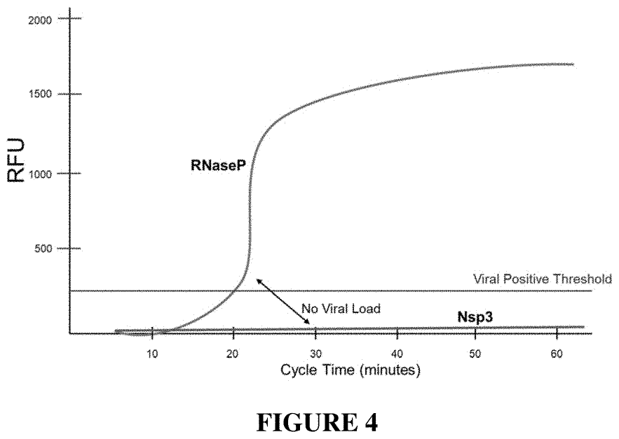

FIG. 4 shows yet more qLAMP results showing amplification resulting from primers directed to SARS-CoV-2 Nsp3-gene cDNA and primers directed to human RNaseP cDNA. In this example, the human RNaseP primers resulted in a threshold breach, but the SARS-CoV-2 Nsp3-gene primers did not. These results indicate no detectable SARS-CoV-2 cDNA in the sample.

The method is performable in less than or equal to about 24 hours, less than or equal to about 12 hours, less than or equal to about 2 hours, or less than or equal to about 1 hour. Accordingly, a subject providing the sample can be determined to have or not have a SARS-CoV-2 infection, i.e., COVID-19, in less than 1 day. This fast turnaround enables periodic monitoring of populations for COVID-19. Exemplary populations include coworkers at a place of employment, students, teachers, and professors at a school, college, or university, citizens of cities, citizens of states, or citizens of nations. Monitoring personal viral loads over time is also enabled by the method.

### II. Methods for Treating COVID-19 in a Subject

The current technology also provides a method for treating COVID-19 in a subject in need thereof. The subject can be a human or non-human mammal having, or suspected of having, COVID-19. The method includes treating the subject with a COVID-19 treatment when a sample taken from the subject is subjected to qLAMP as discussed above and the sample is determined to be positive for SARS-CoV-2. The sample is determined to be positive for SARS-CoV-2 when the sample demonstrates an amplification detection unit threshold breach and/or an amplification rate of greater than or equal to about 50,000 detection units per cycle after RNA from or in the sample is combined with a reverse transcriptase, a DNA polymerase, dNTPs, a marker, and at least two pairs of primers to form a reaction mixture, and the reaction mixture is subjected to qLAMP, wherein the qLAMP generates amplicons comprising a portion of SARS-CoV-2 Nsp3-gene cDNA, a portion of SARS-CoV-2 S-gene cDNA, a portion of a 3′ end of SARS-CoV-2 N-gene cDNA, or a combination thereof.

The COVID-19 treatment can include administering to the subject at least one of an antiviral composition, an anti-inflammatory agent, a steroid, ibuprofen, acetaminophen, a JAK1/JAK2 inhibitor, vitamin D and/or vitamin C, human SARS-CoV-2 antibodies, plasma from a recovered COVID-19 patient, a SARS-CoV-2 antibody, glucocorticoid, aviptadil, famotidine, a neurokinin 1 antagonist, hydroxychloroquine or chloroquine, fluids, or oxygen, as non-limiting examples.

### III. Reaction Mixtures

The current technology also provides a reaction mixture as described above. For example, the reaction mixture can include human mRNA, a DNA polymerase configured for LAMP, a reverse transcriptase, dNTPs, and at least two pairs of primers configured to amplify a portion of SARS-CoV-2 cDNA selected from the group consisting of a portion of Nsp3-gene cDNA, a portion of S-gene cDNA, a portion of a 3′ end of N-gene cDNA, and a combination thereof. The reaction mixture can further include a marker that provides a detectable signal as DNA amplifies, human cDNA, SARS-CoV-2 RNA, SARS-CoV-2 cDNA, or a combination thereof.

In another example, the reaction mixture includes SARS-CoV-2 cDNA, a DNA polymerase configured for LAMP, dNTPs, and greater than or equal to about 100, greater than or equal to about 500, greater than or equal to about 1000, or greater than or equal to about 5000 amplicons corresponding to a portion of SARS-CoV-2 Nsp3-gene cDNA, a portion of SARS-CoV-2 S-gene cDNA, a portion of a 3′ end of SARS-CoV-2 N-gene cDNA, or a combination thereof. Amplicons corresponding to the portion of SARS-CoV-2 Nsp3-gene cDNA can include the sequence as set forth in SEQ ID NO:12. Amplicons corresponding to the portion of SARS-CoV-2 S-gene cDNA can include the sequence as set forth in SEQ ID NO:22. Amplicons corresponding to the portion of SARS-CoV-2 N-gene cDNA can include the sequence as set forth in SEQ ID NO:35. SEQ ID NOs: 12, 22, and 35 are further described in Table 1. The reaction mixture can also include at least one of SARS-CoV-2 RNA, human mRNA, human cDNA, a reverse transcriptase, or at least two pairs of primers configured to amplify a portion of SARS-CoV-2 cDNA selected from the group consisting of a portion Nsp3-gene cDNA, a portion of S-gene cDNA, a portion of a 3′ end of N-gene cDNA, and a combination thereof.

### IV. Diagnostic Assay Kits

The current technology also provides a SARS-CoV-2 assay kit including a primer set, such as

(a) a first primer pair including oligonucleotides having the sequences as set forth in SEQ ID NOs:2 and 3, and a second primer pair including oligonucleotides having the sequences as set forth in SEQ ID NOs:4 and 5,
(b) a primer pair including oligonucleotides having the sequences as set forth in SEQ ID NOs:6 and 7,
(c) a first primer pair including oligonucleotides having the sequences as set forth in SEQ ID NOs:14 and 15, and a second primer pair including oligonucleotides having the sequences as set forth in SEQ ID NOs:16 and 17,
(d) a first primer pair including oligonucleotides having the sequences as set forth in SEQ ID NOs:25 and 26, and a second primer pair including oligonucleotides having the sequences as set forth in SEQ ID NOs:27 and 28,
(e) a primer pair including oligonucleotides having the sequences as set forth in SEQ ID NOs:29 and 30, and/or
(f) a combination thereof.

The diagnostic assay kit can also include control primers configured to amplify a human cDNA. For example the diagnostic assay kit can include human RNaseP control primers including a first control primer pair including first and second control oligonucleotides having the sequences as set forth in SEQ ID NOs:36 and 37, respectively, and a second control primer pair including third and fourth control oligonucleotides having the sequences as set forth in SEQ ID NOs:38 and 39, respectively. The RNaseP control primers can further include a third control primer pair including fifth and sixth oligonucleotides having the sequences as set forth in SEQ ID NOs:40 and 41, respectively.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Example 1. SARS-CoV-2 Detection by LAMP Using Phenol Red

Two clinical samples collected by nasopharyngeal swab were transferred to tubes containing transport medium and delivered to a qLAMP testing facility. At the testing facility, a portion of the transport medium from each sample was transferred to a reaction mixture containing reaction buffer, dNTPs, reverse transcriptase, DNA polymerase, primers, and phenol red. No RNA extractions were performed. The primers were directed to either SARS-CoV-2 Nsp3-gene cDNA and had sequences as set forth in SEQ ID NOs: 2-7, or to human RNaseP and had sequences as set forth in SEQ ID NOs:36-41. A first control included positive (+) single strand of genomic RNA from 2019 Novel Coronavirus (SARS-CoV-2 Isolate USA-WA1/2020|ATCC VR-1986D) as a template and the Nsp3-gene primers. A second control was the same as the first control, but without the template. A third control is an unextracted pseudovirus reference control (AccuPlex™ SARS-CoV-2 Verification Panel-SERACARE) targeted by the Nsp3-gene primers. A fourth control is an unextracted pseudovirus reference control (AccuPlex™ SARS-CoV-2 Verification Panel-SERACARE) targeted by the hRNaseP set of primers. A fifth control included Silt of Puritan® Opti-Swab® Liquid Amies Collection & Transport System added to the isothermal amplification reaction in order to assess any interference with the pH of the reaction and the colorimetric determination. A sixth control included RNAlater Stabilization Solution (Life Technologies Corporation) added to the isothermal amplification reaction in order to assess any interference with the pH of the reaction and the colorimetric determination. The samples and controls were subjected to isothermal amplification at 65° C. for 60 minutes.

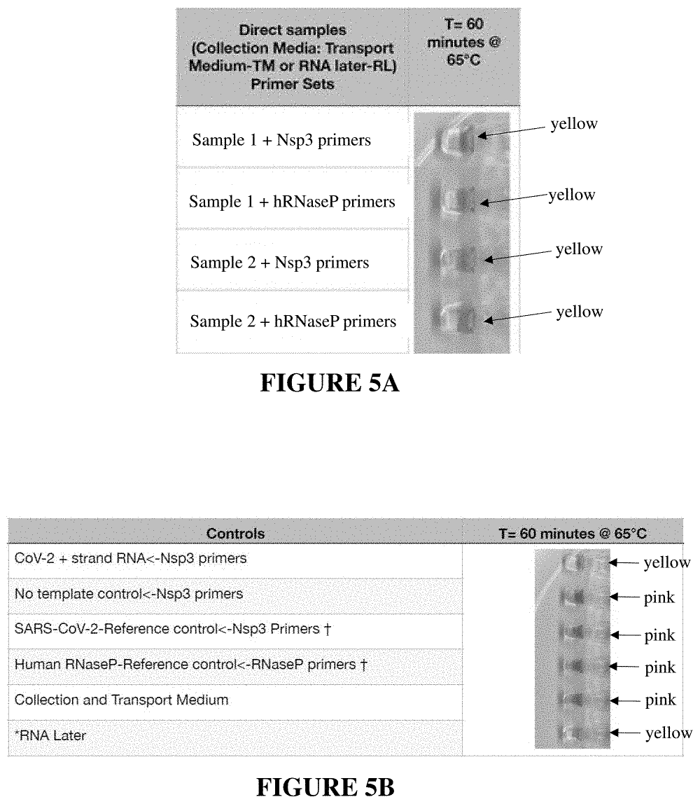

FIG. 5A shows results obtained from the samples. Here, both samples included SARS-CoV-2 RNA as evidenced by the phenol red color changes from pink to yellow resulting from the reactions including Nsp3-gene primers. Both samples also included human mRNA as evidenced by the phenol red color changes from pink to yellow resulting from the reactions including hRNaseP primers.

FIG. 5B shows the results of the controls. Here the first row shows the first control resulting in a positive signal/yellow color. The second row shows the second control resulting in a negative single/pink color. The third row shows the third control resulting in a negative single/pink color. The fourth row shows the fourth control resulting in a negative single/pink color. The fifth row shows the fifth control resulting in a negative/pink color suggesting no interference with the pH of the reaction and the colorimetric determination. The sixth row shows the sixth control resulting in positive/yellow color suggesting interference.

### Example 2. Diluted SARS-CoV-2 Detection by LAMP Using Phenol Red

A standard comprising a known concentration of ATCC® VR1986 SARS-CoV genomic extracted RNA was used to generate samples having 400, 200, 100, 40, and 4 SARS-CoV-2 particles and SARS-CoV-2 Nsp3-gene primers having the sequences as set forth in SEQ ID NOs: 2-7. A first negative control did not include SARS-CoV-2 particles and a second negative control did not include SARS-CoV-2 primers. The samples and controls were denatured at 95° C. for 5 minutes and then subjected to isothermal amplification at 65° C. for 60 minutes.

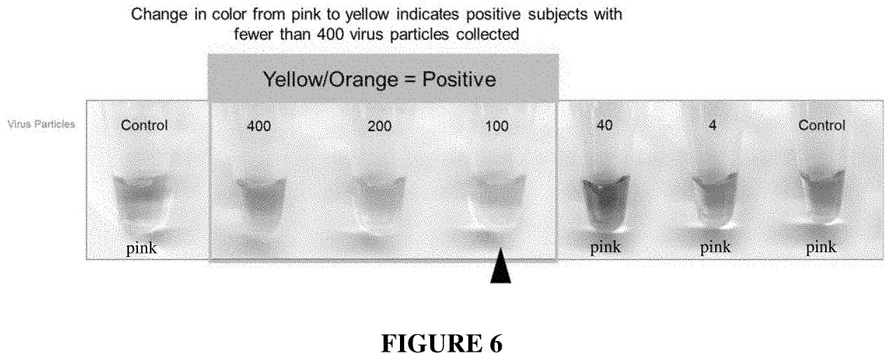

The results are shown in FIG. 6. As can be seen by the phenol red color changes from pink to yellow, the viral loads of 400, 200, and 100 SARS-CoV-2 RNA copies were detectable. The pink color remaining in the samples having viral loads of 40 and 4 particles and the negative controls show that SARS-CoV-2 was not detected in these samples. As shown by the block arrow, a sample having 100 SARS-CoV-2 RNA copies is detectable by the LAMP assay.

### Example 3. Sensitivity of SARS-CoV-2 Detection by LAMP Using Phenol Red

1 μL of Sample 1 from Example 1 from transport medium (positive clinical sample) was transferred to reaction tubes undiluted, and diluted 1:2, 1:4, 1:8, 1:16, 1:32, and 1:64. A negative control included no template (no template control; “NTC”). No RNA extractions were performed. All of the samples included the SARS-CoV-2 Nsp3-gene primers having the sequences as set forth in SEQ ID NOs: 2-7. The samples were denatured at 95° C. for 5 minutes followed by isothermal amplification at 65° C. for 60 minutes.

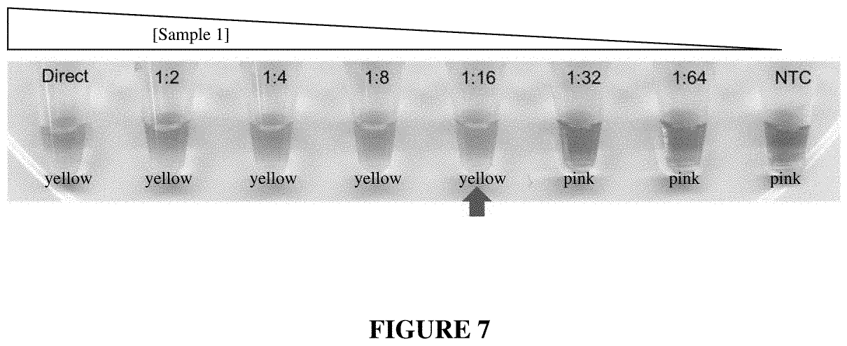

The results are shown in FIG. 7. As can be seen by the phenol red color change from pink to yellow, SARS-CoV-2 was detected in the undiluted sample and the samples diluted 1:2, 1:4, 1:8, and 1:16. SARS-CoV-2 was not detected in the samples diluted 1:32 and 1:64, or in the NTC. Accordingly, the limit of detection (LOD) in this example from the non-extracted RNA of the positive clinical sample is the 1:16 dilution.

### Example 4. SARS-CoV-2 Detection by qLAMP Using Phenol Red and Syto-9

Isothermal amplification reactions were prepared to contain the following templates: direct positive clinical sample, Sample 1 from Example 1 diluted at 1×, 0.5×, 0.2×, 0.1×, and 0, 400 copies of SARS-CoV-2 genomic RNA positive control. 0.4 μM (final) SYTO™ 9 fluorescent marker was added to each reaction. All of the samples included the SARS-CoV-2 Nsp3-gene primers having the sequences as set forth in SEQ ID NOs: 2-7. In addition, a direct SARS-CoV-2 negative clinical sample was set as negative control. Isothermal amplification was performed at 65° C. for 70 minutes.

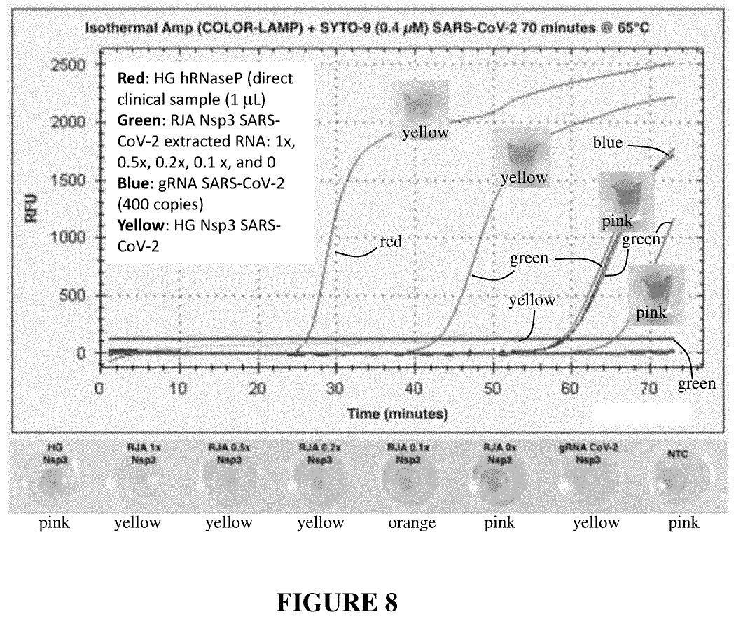

The results are show in FIG. 8. A semi-quantitative real time isothermal amplification of targeted sequences from SARS-CoV-2 viral genome (Nsp3) and human sample loading control (hRNaseP) was assessed by the relative fluorescence signal and compared with their respective colorimetric signal of the reaction tubes. The red line represents the relative amplification of the targeted human RNaseP sample control of the SARS-CoV-2 negative clinical sample but positive human sample amplification. The green lines show relative fluorescent signals from diluted RNA-extracted positive clinical samples. The blue line shows a relative fluorescent signal from Nsp3-targeted SARS-CoV-2 genomic RNA control (Positive amplification control-400 RNA copies/reaction). The yellow line shows a relative fluorescent signal from Nsp3-targeted negative clinical sample control. The results demonstrate a differential threshold of detection favoring the SYTO-9-based semiquantitative fluorescent real time amplification when compared with the colorimetric assessment.

### Example 5. SARS-CoV-2 Detection by qLAMP in Clinical Samples

Clinical samples 1 and 2 described in Example 1 were processed for qLAMP by including 0.4 μM (final) SYTO™ 9 fluorescent marker in each sample along with the SARS-CoV-2 Nsp3-gene primers having the sequences as set forth in SEQ ID NOs: 2-7 and hRNaseP primers having the sequences as set forth in SEQ ID NOs:36-41. A no template control (NTC) was also prepared. Isothermal amplification was performed at 65° C. for 71 minutes.

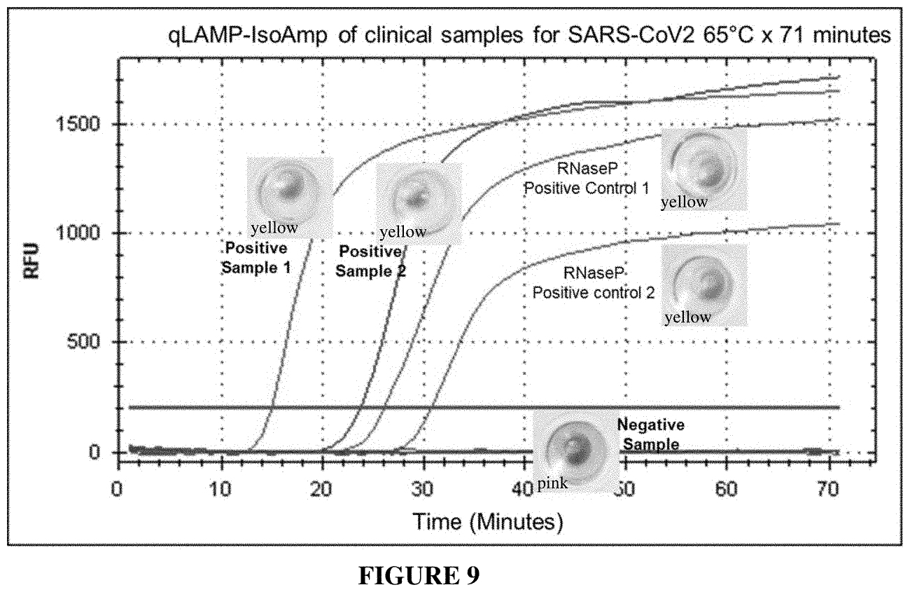

The results are shown in FIG. 9. Here, curves and phenol red color changes from pink to yellow corresponding to Samples 1 and 2 with the SARS-CoV-2 Nsp3-gene primers and hRNaseP primers indicate the presence of SARS-CoV-2 and human mRNA in the samples. The flat curve and lack of color change in the NTC serves as a negative control as expected.

### Example 6. SARS-CoV-2 Detection by qLAMP in Positive Clinical Samples

Positive clinical samples were processed for qLAMP in with SARS-CoV-2 Nsp3-gene primers having the sequences as set forth in SEQ ID NOs: 2-7, SARS-CoV-2 Nucleocapsid-gene primers having the sequences as set forth in SEQ ID NOs: 25-30, and hRNaseP primers. Three negative controls were also prepared. Each PCR tube included phenol red and SYTO™ 9 fluorescent marker. Isothermal amplification was performed at 65° C. for 41 minutes.

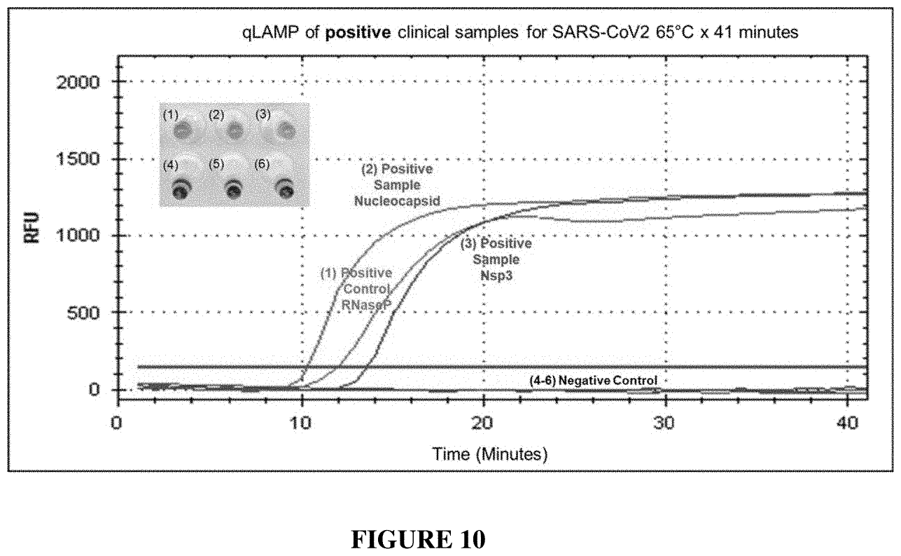

The results are shown in FIG. 10. Here, curves and phenol red color changes from pink to yellow corresponding to samples with the SARS-CoV-2 Nsp3-gene primers, Nucleocapsid-gene primers, and hRNaseP primers indicate the presence of SARS-CoV-2 and human mRNA in the samples. As expected, no amplification was detected in the negative (NTC) controls.

### Example 7. SARS-CoV-2 Determination Using Nsp3-Gene and S-Gene Primers

qLAMP reaction mixtures were prepared having, as a template and primers (template/primers): Nsp3-gene and S-gene control DNA/no primers, SARS-CoV-2 genomic RNA control/Nsp3-gene primers, no template (NTC)/Nsp3-gene primers, SARS-CoV-2 S-gene control DNA/S-gene primers, SARS-CoV-2 RNA/S-gene primers, no template (NTC)/S-gene primers. The SARS-CoV-2 Nsp3-gene primers have the sequences as set forth in SEQ ID NOs: 2-7, and the SARS-CoV-2 S-gene primers have the sequences as set forth in SEQ ID NOs:14-17. Isothermal amplification was performed at 65° C. for 10 minutes and for 45 minutes.

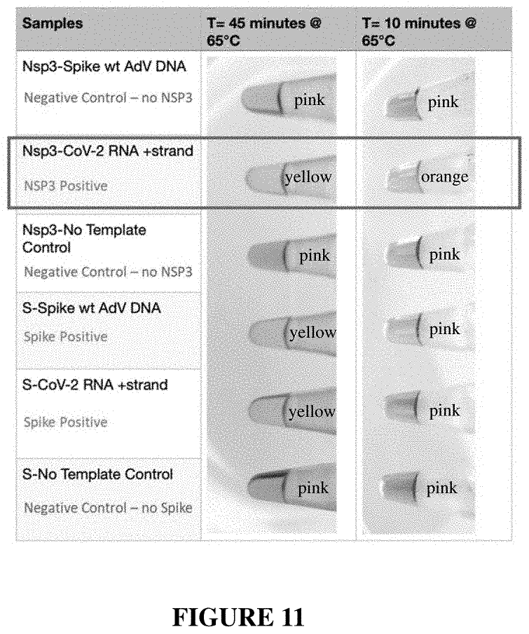

The results are shown in FIG. 11. After 10 minutes, only the reaction mixture containing SARS-CoV-2 genomic RNA control/Nsp3-gene primers generated a pink to orange color shift in phenol red, indicating DNA amplification. After 45 minutes, phenol red pink to yellow transitions are observed in the reaction mixtures including SARS-CoV-2 RNA/Nsp3-gene primers, SARS-CoV-2 S-gene control DNA/S-gene primers, and SARS-CoV-2 RNA/S-gene primers, indicating DNA amplification. As expected, the negative controls did not exhibit DNA amplification.

### Example 8. SARS-CoV-2 Determination Using Nsp3-Gene Primers

qLAMP reaction mixtures were prepared having, as a template and primers (template/primers): SARS-CoV-2 reference DNA (5 μL)/Nsp3-gene primers, SARS-CoV-2 reference DNA (10 μL)/Nsp3-gene primers, human RNaseP control DNA (5 μL)/Nsp3-gene primers, human RNaseP control DNA (10 μL)/Nsp3-gene primers, SARS-CoV-2 RNA (old)/Nsp3-gene primers, SARS-CoV-2 RNA (new)/Nsp3-gene primers, SARS-CoV-2 reference S-gene DNA/Nsp3-gene primers, and no template (NTC)/Nsp3-gene primers. The SARS-CoV-2 Nsp3-gene primers have the sequences as set forth in SEQ ID NOs: 2-7. Isothermal amplification was performed at 65° C. for 45 minutes and for 90 minutes.

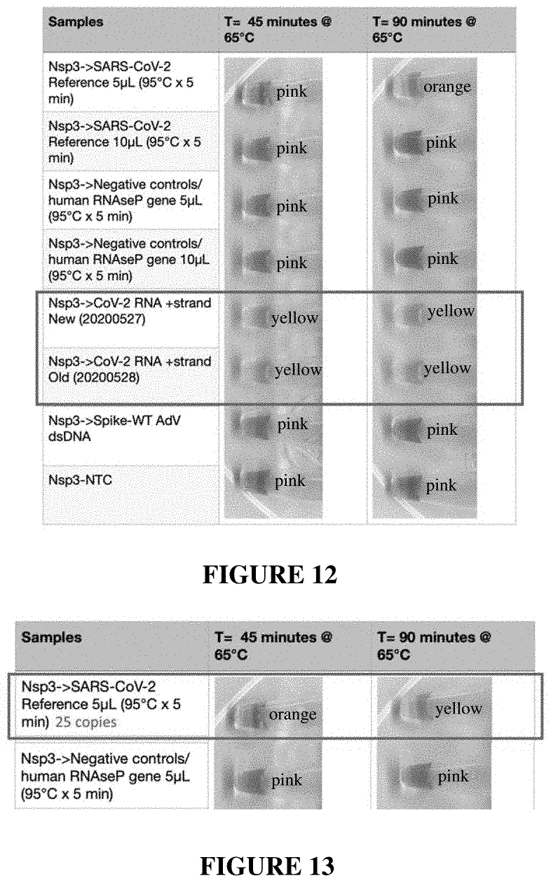

The results are shown in FIG. 12. After 45 and 90 minutes, appreciable amplification is only observed in the reaction mixtures including SARS-CoV-2 RNA (old)/Nsp3-gene primers, SARS-CoV-2 RNA (new)/Nsp3-gene primers, suggesting relative stability and detection at either 45 and 90 minutes.

### Example 9. Sensitivity and LOD from Pseudovirus

A pseudovirus reference control (AccuPlex™ SARS-CoV-2 Verification Panel-SERACARE) was used to verify qLAMP results using SARS-CoV-2 Nsp3-gene primers having the sequences as set forth in SEQ ID NOs: 2-7. A reaction mixture included 25 copies of SARS-CoV-2 genomic DNA and the Nsp3-gene primers and a control reaction mixture included human RNAseP gene DNA and the Nsp3-gene primers. The samples were denatured at 95° C. for 5 minutes followed by isothermal amplification at 65° C. for 45 and 90 minutes.

The results are shown in FIG. 13. As expected, no phenol red color change was observed from the control (NTC) reaction mixtures. Regarding the sample including the SARS-CoV-2 pseudovirus reference control, the phenol red transitioned from pink to orange after 45 minutes and from pink to yellow after 90 minutes. Therefore, the assay can detect a load including 25 copies of SARS-CoV-2 genomic RNA derived from a direct sample with no RNA extraction.

### Example 10. Analysis of Extracted Versus Non-Extracted Samples

Three positive clinical samples were processed for qLAMP with and without (direct) RNA extraction. Each sample included hRNaseP sample control primers. Isothermal amplification was performed at 65° C. for about 70 minutes.

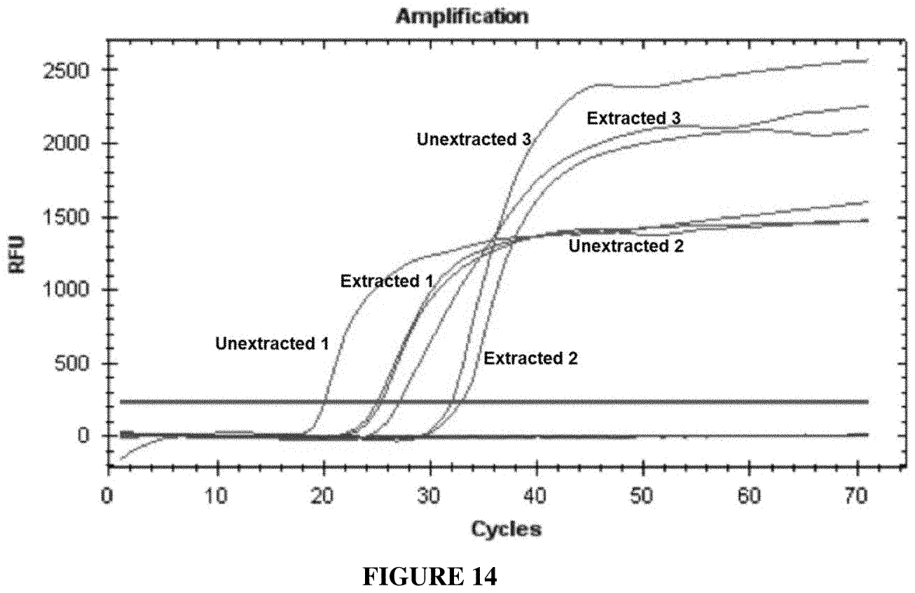

The results are shown in FIG. 14. Here, it can be seen that the cDNA in the non-extracted samples amplified at least as well as the cDNA in the extracted samples.

### Example 11. SARS-CoV-2 Determination in Clinical Samples

A portion of 55 clinical samples obtained from nasopharyngeal swabs were transferred from transfer medium to reaction mixtures including dNTPs, reverse transcriptase, DNA polymerase, and primers. The primers were SARS-CoV-2 Nsp3-gene primers having the sequences as set forth in SEQ ID NOs: 2-7, SARS-CoV-2 N-gene primers having the sequences as set forth in SEQ ID NOs: 25-30, or hRNaseP primers. The reaction mixtures were contained in wells of a 384 well plate having rows A-P and columns 1-24. The samples were subjected to isothermal amplification at 65° C. for 60 minutes.

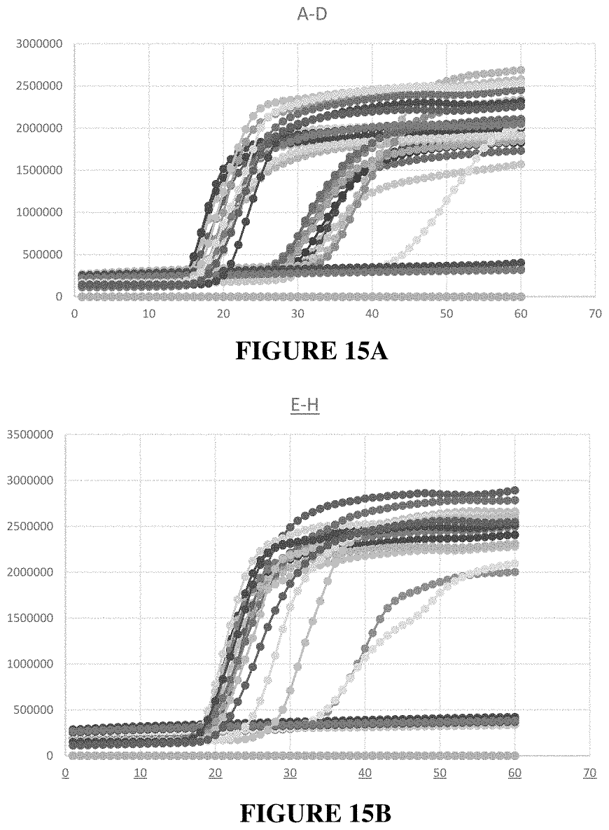

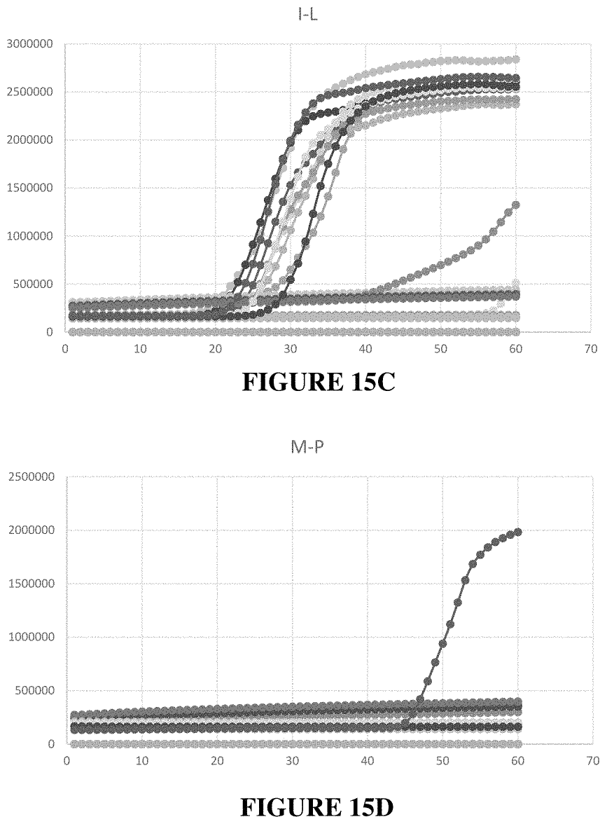

The results are shown in FIGS. 15A-15D. The sigmoidal curves generally show results positive for SARS-CoV-2 and the flat curves generally show results negative for SARS-CoV-2. This example demonstrates that at least 55 clinical samples can be subjected to qLAMP simultaneously with results obtained in about 1 hour. Sigmoidal curves with steep slopes represent relative fluorescent positive signals.

It should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirt and scope of the described invention. All such modifications are intended to be within the scope of the claims appended hereto.

What is claimed is:

1. A method for detecting severe acute respiratory syndrome coronavirus 2 (SARS-CoV-2) in a sample, the method comprising:
contacting at least two pairs of primers with a complementary deoxyribonucleic acid (cDNA) derived from SARS-CoV-2 ribonucleic acid (RNA) in the sample;
amplifying a portion of the cDNA by quantitative loop-mediated isothermal amplification (qLAMP) in the presence of a marker that exhibits a detectable signal as the cDNA is amplified, wherein the portion of the cDNA comprises a portion of non-structural protein 3 (Nsp3)-gene cDNA, a portion of S-gene cDNA, a portion of a 3′ end of N-gene cDNA, or a combination thereof;
periodically measuring the detectable signal during the isothermally amplifying; and
determining at least one of an amplification threshold breach or an amplification rate corresponding to levels of the detectable signal versus amplification time.

2. The method according to claim 1, further comprising reverse transcribing the SARS-CoV-2 RNA to form the cDNA, wherein the SARS-CoV-2 RNA is not extracted from the sample.

3. The method of claim 1, wherein the portion of the cDNA comprises the portion of Nsp3-gene cDNA.

4. The method of claim 1, wherein the portion of the cDNA that is isothermally amplified comprises the portion of N-gene cDNA, and the at least two pairs of primers comprise (1) a first primer pair including first and second oligonucleotides having the sequences as set forth in SEQ ID NOs:2 and 3, respectively, and (2) a second primer pair including third and fourth oligonucleotides having the sequences as set forth in SEQ ID NOs:4 and 5, respectively.

5. The method of claim 4, wherein the at least two pairs of primers further comprise a third primer pair including fifth and sixth oligonucleotides having the sequences as set forth in SEQ ID NOs:6 and 7, respectively.

6. The method of claim 1, wherein the portion of the cDNA that is isothermally amplified comprises the portion of S-gene cDNA, and the at least two pairs of primers comprise (1) a first primer pair including first and second oligonucleotides having the sequences as set forth in SEQ ID NOs:14 and 15, respectively, and (2) a second primer pair including third and fourth oligonucleotides having the sequences as set forth in SEQ ID NOs:16 and 17, respectively.

7. The method of claim 1, wherein the portion of the cDNA that is isothermally amplified comprises the portion of N-gene cDNA, and the at least two pairs of primers comprise (1) a first primer pair including first and second oligonucleotides having the sequences as set forth in SEQ ID NOs:25 and 26, respectively, and (2) a second primer pair including third and fourth oligonucleotides having the sequences as set forth in SEQ ID NOs:27 and 28, respectively.

8. The method of claim 7, wherein the at least two pairs of primers further comprise a third primer pair including fifth and sixth oligonucleotides having the sequences as set forth in SEQ ID NOs:29 and 30, respectively.

9. The method of claim 1, wherein the isothermally amplifying is performed for less than or equal to about 60 minutes.

10. The method of claim 1, wherein a viral load of about 1000 copies/mL or greater in the sample is quantitatively measured.

11. A method for treating corona virus disease 2019 (COVID-19) in a subject in need thereof, the method comprising:
treating the subject with a COVID-19 treatment when a sample taken from the subject demonstrates an amplification detection unit threshold breach and an amplification rate of greater than or equal to about 50,000 detection units per cycle after RNA from or in the sample is combined with a reverse transcriptase, a deoxyribonucleic acid (DNA) polymerase, deoxyribonucleotide triphosphates (dNTPs), a marker, and at least two pairs of primers to form a reaction mixture, and the reaction mixture is subjected to quantitative loop-mediated isothermal amplification (qLAMP),
wherein the qLAMP generates amplicons comprising a portion of SARS-CoV-2 non-structural protein 3 (Nsp3)-gene cDNA, a portion of SARS-CoV-2 S-gene cDNA, a portion of a 3′ end of SARS-CoV-2 N-gene cDNA, or a combination thereof.

12. The method of claim 11, wherein the at least two pairs of primers comprise (1) a first primer pair including first and second oligonucleotides having the sequences as set forth in SEQ ID NOs:2 and 3, respectively, (2) a second primer pair including third and fourth oligonucleotides having the sequences as set forth in SEQ ID NOs:4 and 5, respectively, and (3) a third primer pair including fifth and sixth oligonucleotides having the sequences as set forth in SEQ ID NOs:6 and 7, respectively.

13. The method of claim 11, wherein the at least two pairs of primers comprise (1) a first primer pair including first and second oligonucleotides having the sequences as set forth in SEQ ID NOs:14 and 15, respectively, and (2) a second primer pair including third and fourth oligonucleotides having the sequences as set forth in SEQ ID NOs:16 and 17, respectively.

14. The method of claim 11, wherein the at least two primers comprise (1) a first primer pair including first and second oligonucleotides having the sequences as set forth in SEQ ID NOs:25 and 26, respectively, (2) a second primer pair including third and fourth oligonucleotides having the sequences as set forth in SEQ ID NOs:27 and 28, respectively, and (3) a third primer pair including fifth and sixth oligonucleotides having the sequences as set forth in SEQ ID NOs:29 and 30, respectively.

15. A reaction mixture comprising:
human messenger ribonucleic acid (mRNA);
a deoxyribonucleic acid (DNA) polymerase configured for loop-mediated isothermal amplification (LAMP);
a reverse transcriptase;
deoxyribonucleotide triphosphates (dNTPs); and
at least two pairs of primers configured to amplify a portion of severe acute respiratory syndrome coronavirus 2 (SARS-CoV-2) complementary DNA (cDNA) selected from the group consisting of a portion non-structural of protein 3 (Nsp3)-gene cDNA, a portion of S-gene cDNA, a portion of a 3′ end of N-gene cDNA, and a combination thereof.

16. The reaction mixture of claim 15, further comprising a marker that provides a detectable signal as DNA amplifies.

17. The reaction mixture of claim 15, further comprising SARS-CoV-2 RNA.

18. The reaction mixture of claim 15, wherein the at least two pairs of primers comprise (1) a first primer pair including first and second oligonucleotides having the sequences as set forth in SEQ ID NOs:2 and 3, respectively, (2) a second primer pair including third and fourth oligonucleotides having the sequences as set forth in SEQ ID NOs:4 and 5, respectively, and (3) a third primer pair including fifth and sixth oligonucleotides having the sequences as set forth in SEQ ID NOs:6 and 7, respectively.

19. The reaction mixture of claim 15, wherein the at least two pairs of primers comprise (1) a first primer pair including first and second oligonucleotides having the sequences as set forth in SEQ ID NOs:14 and 15, respectively, and (2) a second primer pair including third and fourth oligonucleotides having the sequences as set forth in SEQ ID NOs:16 and 17, respectively.

20. The reaction mixture of claim 15, wherein the at least two pairs of primers comprise (1) a first primer pair including first and second oligonucleotides having the sequences as set forth in SEQ ID NOs:25 and 26, respectively, (2) a second primer pair including third and fourth oligonucleotides having the sequences as set forth in SEQ ID NOs:27 and 28, respectively, and (3) a third primer pair including fifth and sixth oligonucleotides having the sequences as set forth in SEQ ID NOs:29 and 30, respectively.
